(19) United States
(12) Patent Application Publication
(10) Pub. No.: US 2004/0072801 A1
(43) Pub. Date: Apr. 15, 2004

(54) Sterols bearing pendant allosteric effectors of hemoglobin, and uses thereof

(76) Inventors: Yves Claude Nicolau, Newton, MA (US); Jean-Marie Lehn, Strasbourg (FR); Konstantina Fylaktakidou, Alexandroupolis (GR); Stephane P. Vincent, Paris (FR)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/459,661

(22) Filed: Jun. 11, 2003

Related U.S. Application Data
(60) Provisional application No. 60/388,851, filed on Jun. 14, 2002.

(51) Int. Cl. A61K 31/685; C07J 1/00

(52) U.S. Cl. 514/78; 552/506

(57) ABSTRACT

The present invention comprises compounds, compositions thereof, and methods capable of delivering a broad range of anionic molecules to the cytoplasm of mammalian cells. In certain embodiments, the present invention relates to compounds, compositions thereof, and methods that enhance the ability of mammalian red blood cells to deliver oxygen, by delivering a ligand for the allosteric site of hemoglobin to the cytoplasm of the red blood cells.

### RELATED APPLICATIONS

This application claims the benefit of priority to U.S. Provisional Patent Application serial No. 60/388,851, filed Jun. 14, 2002.

### BACKGROUND OF THE INVENTION

I. Ischemia

Ischemic insult, i.e., the localized deficiency of oxygen to an organ or skeletal tissue, is a common and important problem in many clinical conditions. The problem is especially acute in organ transplant operations in which a harvested organ is removed from a body, isolated from a blood source, and thereby deprived of oxygen and nutrients for an extended period of time. Ischemic insult also occurs in certain clinical conditions, such as sickle cell anemia and septic shock, which may result from hypotension or organ dysfunction. Depending on the duration of the insult, the ischemia can disturb cellular metabolism and ion gradients, and ultimately cause irreversible cellular injury and death.

Arguably, heart attacks and stroke are the most widely recognized example of the damage resulting from ischemia. Myocardial ischemia is a condition wherein there is insufficient blood supply to the myocardium (the muscles of the heart) to meet its demand for oxygen. The ultimate result of persistent myocardial ischemia is necrosis or death of a portion of cardiac muscle tissue, known as a myocardial infarct, commonly known as a heart attack. Insufficient blood supply to the myocardium is generally due to an obstruction or thrombus in an artery supplying blood to the myocardium. Another cause can be atrial fibrillation, wherein the increased heart rate associated with atrial fibrillation increases the work, and hence the blood demand of the myocardium, while the atrial fibrillation at the same time reduces the blood supply.

Stroke is defined as a sudden impairment of body functions caused by a disruption in the supply of blood to the brain. For instance, a stroke occurs when blood supply to the brain is interrupted for any reason, including hemorrhage, low blood pressure, clogging by atherosclerotic plaque, a blood clot, or any particle. Because of the blockage or rupture, part of the brain fails to get the supply of blood and oxygen that it requires. Brain tissue that receives an inadequate supply of blood is said to be ischemic. Deprived of oxygen and nutrients, nerve cells and other cell types within the brain begin to fail, creating an infarct (an area of cell death, or necrosis). As the neurons fail and die, the part of the body controlled by those neurons can no longer function. The devastating effects of ischemia are often permanent because brain tissue has very limited repair capabilities and lost neurons are typically not regenerated.

Cerebral ischemia may be incomplete (blood flow is reduced but not entirely cut off), complete (total loss of tissue perfusion), transient or permanent. If ischemia is incomplete and persists for no more than ten to fifteen minutes, neural death may not occur. More prolonged or complete ischemia results in infarction. Depending on the site and extent of the infarction, mild to severe neurological disability or death will follow.

To a modest extent, the brain is protected against cerebral ischemia by compensatory mechanisms, including collateral circulation (overlapping local blood supplies), and arteriolar auto-regulation (local smooth muscle control of blood flow in the smallest arterial channels). If compensatory mechanisms operate efficiently, slightly diminished cerebral blood flow produces neither tissue ischemia nor abnormal signs and symptoms. Usually, such mechanisms must act within minutes to restore blood flow if permanent infarction damage is to be avoided or reduced. Arteriolar auto-regulation works by shunting blood from noncritical regions to infarct zones. Even in the face of systemic hypotension, auto-regulation may be sufficient to adjust the circulation and thereby preserve the vitality and function of brain or heart tissue. Alternatively, ischemia may be sufficiently prolonged and compensatory mechanisms sufficiently inadequate that a catastrophic stroke or heart attack results.

Ischemia is also associated with various clinical conditions, such as septic shock. Septic shock as a result of hypotension and organ dysfunction in response to infectious sepsis is a major cause of death. The manifestations of sepsis include those related to the systemic response to infection (tachycardia, tachypnea alterations in temperature and leukocytosis) and those related to organ-system dysfunction (cardiovascular, respiratory, renal, hepatic and hematologic abnormalities). Furthermore, the lipopolysaccharide (LPS) of Gram-negative bacteria is considered to be the most important exogenous mediator of acute inflammatory response to septic shock. The LPS or endotoxin released from the outer membrane of Gram-negative bacteria results in the release of cytokines and other cellular mediators, including tumor necrosis factor alpha (TNF alpha), interleukin-1 (II-1), interleukin-6 (II-6) and thromboxane A2. Extreme levels of these mediators are known to trigger many pathological events, including fever, shock, and intravascular coagulation, leading to ischemia and organ failure.

II. Hemoglobin

Hemoglobin is a tetrameric protein which delivers oxygen via an allosteric mechanism. Oxygen binds to the four hemes of the hemoglobin molecule. Each heme contains porphyrin and iron in the ferrous state. The formation of the ferrous iron-oxygen bond is readily reversible. Binding of the first oxygen to a heme releases much greater energy than binding of the second oxygen molecule, binding of the third oxygen releases even less energy, and binding of the fourth oxygen releases the least energy.

In blood, hemoglobin is in equilibrium between two allosteric structures. In the “T” (for tense) state, hemoglobin is deoxygenated. In the “R” (for relaxed) state, hemoglobin is oxygenated. An oxygen equilibrium curve can be scanned to observe the affinity and degree of cooperativity (allosteric action) of hemoglobin. In the scan, the Y-axis plots the percent of hemoglobin oxygenation and the X-axis plots the partial pressure of oxygen in millimeters of mercury (mm Hg). If a horizontal line is drawn from the 50% oxygen saturation point to the scanned curve and a vertical line is drawn from the intersection point of the horizontal line with the curve to the partial pressure X-axis, a value commonly known as the P₅₀ is determined (i.e., the pressure in mm Hg when the scanned hemoglobin sample is 50% saturated with oxygen). Under physiological conditions (i.e., 37 C, pH=7.4, and partial carbon dioxide pressure of 40 mm Hg), the P₅₀ value for normal adult hemoglobin (HbA) is around 26.5 mm Hg. If a lower than normal P₅₀ value is obtained for the hemoglobin being tested, the scanned curve is considered to be “left-shifted” and the presence of high oxygen-affinity hemoglobin is indicated. Conversely, if a higher than normal P₅₀ value is obtained for the hemoglobin being tested, the scanned curve is considered to be “right-shifted”, indicating the presence of low oxygen-affinity hemoglobin.

It has been proposed that influencing the allosteric equilibrium of hemoglobin is a viable avenue of attack for treating diseases. The conversion of hemoglobin to a high affinity state is generally regarded to be beneficial in resolving problems with (deoxy)hemoglobin-S (i.e., sickle cell anemia). The conversion of hemoglobin to a low affinity state is believed to have general utility in a variety of disease states where tissues suffer from low oxygen tension, such as ischemia and radio sensitization of tumors. Several synthetic compounds have been identified which have utility in the allosteric regulation of hemoglobin and other proteins. For example, several new compounds and methods for treating sickle cell anemia which involve the allosteric regulation of hemoglobin are reported in U.S. Pat. No. 4,699,926 to Abraham et al., U.S. Pat. No. 4,731,381 to Abraham et al., U.S. Pat. No. 4,731,473 to Abraham et al., U.S. Pat. No. 4,751,244 to Abraham et al., and U.S. Pat. No. 4,887,995 to Abraham et al. Furthermore, in both Perutz, “Mechanisms of Cooperativity and allosteric Regulation in Proteins”, Quarterly Reviews of Biophysics 22, 2 (1989), pp. 163-164, and Lalezari et al., “LR16, a compound with potent effects on the oxygen affinity of hemoglobin, on blood cholesterol, and on low density lipoprotein”, Proc. Natl. Acad. Sci., USA 85 (1988), pp. 6117-6121, compounds which are effective allosteric hemoglobin modifiers are discussed. In addition, Perutz et al. has shown that a known antihyperlipoproteinemia drug, bezafibrate, is capable of lowering the affinity of hemoglobin for oxygen (See “Bezafibrate lowers oxygen affinity of hemoglobin”, Lancet 1983, 881).

Normal human adult hemoglobin (“HbA”) is a tetrameric protein containing two alpha chains having 141 amino acid residues each and two beta chains having 146 amino acid residues each, and also bearing prosthetic groups known as hemes. The erythrocytes help maintain hemoglobin in its reduced, functional form. The heme-iron atom is susceptible to oxidation, but may be reduced again by one of two systems, the cytochrome b5 system and the glutathione reduction system, within the erythrocyte.

Hemoglobin is able to alter its oxygen affinity, thereby increasing the efficiency of oxygen transport in the body due to its dependence on an allosteric regulator, 2,3-DPG. 2,3-DPG is present within erythrocytes at a concentration that facilitates hemoglobin to release bound oxygen to tissues. Naturally-occurring hemoglobin includes any hemoglobin identical to hemoglobin naturally existing within a cell. Naturally-occurring hemoglobin is predominantly wild-type hemoglobin, but also includes naturally-occurring mutant hemoglobin. Wild-type hemoglobin is hemoglobin most commonly found within natural cells. Wild-type human hemoglobin includes hemoglobin A, the normal adult human hemoglobin having two alpha- and two beta-globin chains. Mutant hemoglobin has an amino-acid sequence that differs from the amino-acid sequence of wild-type hemoglobin as a result of a mutation, such as a substitution, addition or deletion of at least one amino acid. Adult human mutant hemoglobin has an amino-acid sequence that differs from the amino-acid sequence of hemoglobin A. Naturally-occurring mutant hemoglobin has an amino-acid sequence that has not been modified by humans. The naturally-occurring hemoglobin of the present invention is not limited by the methods by which it is produced. Such methods typically include, for example, erythrocytolysis and purification, recombinant production, and protein synthesis.

Hemoglobin specifically binds various small polyanionic molecules, including 2,3-diphosphoglycerate (DPG) and adenosine triphosphate (ATP), present in mammalian red blood cells (Benesch and Benesch, Nature, Vol. 221, p. 618, 1969). The binding site is located at the center of the tetrameric structure of hemoglobin (Arnone, A., Nature, Vol. 237, p. 146, 1972). The binding of these polyanionic molecules is important in regulating the oxygen-binding affinity of hemoglobin since it allosterically affects the conformation of hemoglobin leading to a decrease in oxygen affinity (Benesch and Benesch, Biochem. Biophys. Res. Comm., Vol. 26, p. 162, 1967). Conversely, the binding of oxygen allosterically reduces the affinity of hemoglobin for the polyanion. Therefore, (oxy)hemoglobin binds DPG and ATP weakly. This is shown, for example, by studies of spin-labeled ATP binding to oxy- and deoxyhemoglobin as described by Ogata and McConnell (Ann. N.Y. Acad. Sc., Vol. 222, p. 56, 1973). In order to exploit the polyanion-binding specificity of hemoglobin, or indeed to perform any adjustment of its oxygen-binding affinity by chemically modifying the polyanion binding site, it has been necessary in the prior art that hemoglobin be deoxygenated. However, hemoglobin as it exists in solutions, or mixtures exposed to air, is in its oxy state, i.e., (oxy)hemoglobin. In fact it is difficult to maintain hemoglobin solutions in the deoxy state, (deoxy)hemoglobin, throughout a chromatographic procedure.

Hemoglobin has also been administered as a pretreatment to patients receiving chemotherapeutic agents or radiation for the treatment of tumors (U.S. Pat. No. 5,428,007; WO 92/20368; WO 92/20369), for prophylaxis or treatment of systemic hypotension or septic shock induced by internal nitric oxide production (U.S. Pat. No. 5,296,466), during the perioperative period or during surgery in a method for maintaining a steady-state hemoglobin concentration in a patient (WO 95/03068), and as part of a perioperative hemodilution procedure used prior to surgery in an autologous blood use method (U.S. Pat. Nos. 5,344,393 and 5,451,205). When a patient suffers a trauma (i.e., a wound or injury) resulting, for example, from surgery, an invasive medical procedure, or an accident, the trauma disturbs the patient's homeostasis. The patient's body biologically reacts to the trauma to restore homeostasis. This reaction is referred to herein as a naturally occurring stress response. If the body's stress response is inadequate or if it occurs well after the trauma is suffered, the patient is more prone to develop disorders.

III. Reduction of the Oxygen-Affinity of Hemoglobin

The major function of erythrocytes consists in the transport of molecular oxygen from the lungs to the peripheral tissues. The erythrocytes contain a high concentration of hemoglobin (30 pg per cell=35.5 g/100 ml cells) which forms a reversible adduct with O₂. The O₂-partial pressure in the lung is about 100 mm Hg, in the capillary system is about 70 mm Hg, against which O₂ must be dissociated from the oxygenated hemoglobin. Under physiological conditions, only about 25% of the oxygenated hemoglobin may be deoxygenated; about 75% is carried back to the lungs with the venous blood. Thus, the major fraction of the hemoglobin-O₂ adduct is not used for the O₂ transport.

Interactions of hemoglobin with allosteric effectors enable an adaptation to the physiological requirement of maximum O₂ release from the hemoglobin-O₂ adduct with simultaneous conservation of the highest possible O₂ partial pressure in the capillary system. 2,3-Diphosphoglycerate increases the half-saturation pressure of stripped hemoglobin at pH 7.4 from P(O₂)(½)=9.3 mm Hg (37 C), and 4.3 mm Hg (25 C) to P(O₂)(½)=23.7 mm Hg (37 C), and 12.0 mm Hg (25 C), respectively (Imai, K. and Yonetani, T. (1975), J. Biol. Chem. 250, 1093-1098). A significantly stronger decrease of the O₂ affinity, i.e., enhancement of the O₂ half-saturation pressure has been achieved for stripped hemoglobin by binding of inositol hexaphosphate (phytic acid; IHP) (Ruckpaul, K. et al. (1971) Biochim. Biophys. Acta 236, 211-221) isolated from vegetal tissues. Binding of IHP to hemoglobin increases the O₂ half-saturation pressure to P(O₂)(½)=96.4 mm Hg (37 C), and P(O₂)(½)=48.4 mm Hg (25 C), respectively. IHP, like 2,3-diphosphoglycerate and other polyphosphates, cannot penetrate the erythrocyte membrane.

Furthermore, the depletion of DPG and ATP in stored red cells leads to a progressive increase of the oxygen affinity of hemoglobin contained therein (Balcerzak, S. et al. (1972) Adv. Exp. Med. Biol. 28, 453-447). The O₂-binding isotherms are measured in the absence of CO₂ and at constant pH (pH 7.4) in order to preclude influences of these allosteric effectors on the half-saturation pressure. The end point of the progressive polyphosphate depletion is defined by P(O₂)(½)=4.2 mm Hg, which is the half-saturation pressure of totally phosphate-free (stripped) hemoglobin; the starting point, i.e., P(O₂) (½) of fresh erythrocytes, depends on the composition of the suspending medium. From these polyphosphate depletion curves a new functional parameter of stored erythrocytes can be determined, the so-called half-life time of intraerythrocytic polyphosphate: 9 d (days) in isotonic 0.1 M bis-Tris buffer pH 7.4; and 12 d (days) in acid-citrate-dextrose conservation (ACD) solution.

Several years ago, it was discovered that the antilipidemic drug clofibric acid lowered the oxygen affinity of hemoglobin solutions (Abraham et al., J. Med. Chem. 25, 1015 (1982), and Abraham et al., Proc. Natl. Acad. Sci. USA 80, 324 (1983)). Bezafibrate, another antilipidemic drug, was later found to be much more effective in lowering the oxygen affinity of hemoglobin solutions and suspensions of fresh, intact red cells (Perutz et al., Lancet, 881, Oct. 15, 1983). Subsequently, X-ray crystallographic studies have demonstrated that clofibric acid and bezafibrate bind to the same sites in the central water cavity of deoxyhemoglobin, and that one bezafibrate molecule will span the sites occupied by two clofibric acid molecules. Bezafibrate and clofibric acid act by stabilizing the deoxy structure of hemoglobin, shifting the allosteric equilibrium toward the low affinity deoxy form. Bezafibrate and clofibric acid do not bind in any specific manner to either oxy- or carbonmonoxyhemoglobin.

In more recent investigations, a series of urea derivatives [2-[4-[[(arylamino)carbonyl]amino]phenoxy]-2-methylpropionic acids] was discovered that has greater allosteric potency than bezafibrate at stabilizing the deoxy structure of hemoglobin and shifting the allosteric equilibrium toward the low oxygen affinity form (Lalezari, Proc. Natl. Acad. Sci. USA 85, 6117 (1988)).

Drugs which can allosterically modify hemoglobin toward a lower oxygen affinity state hold potential for many clinical applications, such as for the treatment of ischemia, shock, and polycythemia, and as radiosensitizing agents. Unfortunately, the effects of bezafibrate and the urea derivatives discussed above have been found to be significantly inhibited by serum albumin, the major protein in blood serum (Lalezari et al., Biochemistry, 29, 1515 (1990)). Therefore, the clinical usefulness of these drugs is seriously undermined because in whole blood and in the body, the drugs would be bound by serum albumin instead of reaching the red cells, crossing the red cell membrane, and interacting with hemoglobin protein molecule to produce the desired effect.

There has been considerable interest in medicine, the military health services, and the pharmaceutical industry in finding methods to increase oxygen delivery in vivo for ischemic insults, stroke, and trauma; to increase blood storage life; to discover radio sensitization agents; and to develop new blood substitutes. In all these instances, the availability of either autologous blood or recombinant Hb solutions is of major interest, provided the oxygen affinity can be decreased to enhance oxygen delivery to the tissues.

2,3-Diphosphoglycerate (2,3-DPG) is the normal physiological ligand for the allosteric site on hemoglobin. However, phosphorylated inositols are found in the erythrocytes of birds and reptiles. Specifically, inositol hexaphosphate (IHP), as known as phytic acid, displaces hemoglobin-bound 2,3-DPG, binding to the allosteric site with one-thousand times greater affinity. Unfortunately, IHP is unable to pass unassisted across the erythrocyte membrane.

IV. Enhanced Oxygen Delivery in Mammals

The therapy of oxygen deficiencies requires the knowledge of parameters which characterize both the O₂ transport capacity and the O₂ release capacity of human RBCs. The parameters of the O₂ transport capacity, i.e., Hb concentration, the number of RBCs, and hemocrit, are commonly used in clinical diagnosis. However, the equally important parameters of the O₂ release capacity, i.e., O₂ half-saturation pressure of Hb and RBCs, and the amounts of high and low oxygen affinity hemoglobins in RBCs, are not routinely determined and were not given serious consideration until pioneering work by Gerosonde and Nicolau (Blut, 1979, 39, 1-7).

In the 1980s, Nicolau et al. (J. Appl. Physiol. 58:1810-1817 (1985); “PHYTIC ACID: Chemsitry and Applications”; Graf, E., Ed.; Pilatus Press, Minneapolis, Minn., USA; 1986; and Proc. Natl. Acad. Sci. USA 1987, 84, 6894-6898) reported that the encapsulation in red blood cells (RBCs) of IHP, via a technique of controlled lysis and resealing, results in a significant decrease in the hemoglobin affinity for oxygen. The procedure yielded RBCs with unchanged life spans, normal ATP and K+ levels, and normal rheological competence. Enhancement of the O₂-release capacity of these cells brought about significant physiological effects in piglets: 1) reduced cardiac output, linearly dependent on the P₅₀ value of the RBCs; 2) increased arteriovenous difference; and 3) improved tissue oxygenation. Long term experiments showed that in piglets the high P₅₀ value of IHP-RBCs was maintained over the entire life spans of the RBCs.

More recently, Nicolau et al. (TRANSFUSION 1995, 35, 478-486; and U.S. Pat. No. 5,612,207) reported the use of a large-volume, continuous-flow electroporation system for the encapsulating IHP in human RBCs. These modified RBCs possess P₅₀ values of approximately 50 torr, roughly twice that of unmodified human RBCs. Additionally, 85% of the RBCs survived the electroporation process, displaying hematologic indices nearly identical to those of unmodified RBCs. Nicolau's electroporation system processes one unit of blood every ninety minutes.

### SUMMARY OF THE INVENTION

The present invention relates to compositions, and methods of use thereof, consisting essentially of lipophilic moieties covalently bound to an allosteric effector, i.e., ligand for the allosteric site, of hemoglobin, e.g., inositol hexaphosphate (IHP).

In certain embodiments, the present invention relates to compounds, and compositions thereof, that deliver into erythrocytes allosteric effectors of hemoglobin in vivo. Additionally, the invention is directed to the use of these compounds, or compositions thereof, for lowering the oxygen affinity of hemoglobin in red blood cell suspensions and whole blood. It is an object of this invention to provide methods for delivering into erythrocytes allosteric effectors of hemoglobin in whole blood and in vivo, utilizing compounds, or compositions thereof, that do not lose their effectiveness in the presence of normal concentrations of the remaining components of whole blood.

Ligands for the allosteric site of hemoglobin interact with the hemoglobin molecule and impact its ability to bind oxygen. This invention is particularly concerned with the delivery into erythrocytes of ligands for the hemoglobin allosteric site, causing oxygen to be bound relatively less tightly to hemoglobin, such that oxygen is off-loaded from the hemoglobin molecule more easily.

The process of allosterically modifying hemoglobin towards a lower oxygen affinity state in whole blood and in vivo may be used in a wide variety of applications, including treatments for ischemia, heart disease, wound healing, radiation therapy of cancer, and adult respiratory distress syndrome (ARDS). Furthermore, a decrease in the oxygen affinity of hemoglobin in whole blood will extend its useful shelf-life vis-à-vis transfusions, and/or restore the oxygen carrying capacity of aged blood.

### BRIEF DESCRIPTION OF THE FIGURES

FIG. 1 depicts inositol hexaphosphate (IHP) covalently bonded to a cholesterol moiety through an oxycarbonyl linking group.

FIG. 2 depicts a shift in P50 curves for SV184 versus a saline control. Curve A: Control (saline), P₅₀=33.3; Curve B: SV184 IV. WB mouse, 1 h, P₅₀=41.0, % increase=23.12.

FIG. 3 depicts the structure of KF16—Inositol hexaphosphate-cholesteroloxycarbonyl, hepta N,N-dimethyl-cyclohexylammonium salt.

FIG. 4 depicts the P50 shift in blood of C57BL/6 mice injected with KF16.3 (45 mM) as a function of time.

FIG. 5 depicts the P50 shift in mice RBC after intraperitonal injection of KF16.4 at various concentrations.

### DETAILED DESCRIPTION OF THE INVENTION

I. Overview

The process of allosterically modifying hemoglobin towards a low oxygen affinity state in whole blood and in vivo could be used in a wide variety of applications including in treatments for ischemia, heart disease, complications associated with angioplasty, wound healing, radiation therapy of cancer, adult respiratory distress syndrome (ARDS), etc., in extending the shelf-life of blood or restoring the oxygen carrying capacity of out-dated blood, and as sensitizers for x-ray irradiation in cancer therapy, as well as in many other applications.

This invention is related to the delivery of allosteric hemoglobin modifier compounds into red blood cells, in whole blood and in vivo, using the compounds of the present invention Serum albumin, which is the most abundant protein in blood plasma, has been identified as inhibiting the allosteric effects of clofibric acid, bezafibrate, and L3,5/L3,4,5. The precise nature of this inhibition is not fully understood, but appears to be related to these compounds binding to the serum albumin. In contrast, the subject compounds have been found to be relatively unaffected by the presence of serum albumin. Ligands for the allosteric site of hemoglobin that are not adversely effected by serum albumin represent particularly good candidates for drug applications, since the performance of the drug will not be frustrated by the presence of serum albumin present in a patient's blood.

This invention relates to compounds that facilitate the delivery of a wide variety of therapeutically useful substances into mammalian red blood cells (RBCs). In particular, the compounds and methods of the present invention make possible the introduction or incorporation into RBCs of anionic agents, such as allosteric effectors of hemoglobin, DNA, RNA, chemotherapeutic agents, and antibiotic agents.

The present invention provides novel compounds and methods for increasing the oxygen-carrying capacity of erythrocytes. In accordance with the present invention, the allosteric effector, e.g., IHP, is delivered into the RBCs where it combines with hemoglobin, and shifting its oxygen releasing capacity. Erythrocytes with IHP-hemoglobin can release more oxygen per molecule than hemoglobin alone, and thus more oxygen is available to diffuse into tissues for each unit of blood that circulates. Critically, when injected in vivo, IHP is toxic, i.e., it cannot be tolerated as an ordinary drug.

Another advantage of IHP-treated red blood cells is that they show the Bohr effect in circulation and when stored. Normal red blood cells that have been stored do not regain their maximum oxygen carrying capacity in circulation for approximately 24 hours because the DPG present in normal red blood cells is degraded by native enzymes, e.g., phosphatases, during storage and must be replaced by the body after transfusion. In contrast, red blood cells treated according to the present invention retain their maximum oxygen carrying capacity during storage because IHP is not degraded by the aforementioned enzymes; therefore, they can deliver oxygen to the tissues in response to demand immediately after transfusion into a human or animal, i.e., because there are no native enzymes in erythrocytes which degrade IHP.

IHP-treated RBCs may be used in the treatment of acute and chronic conditions, including, but not limited to, hospitalized patients, cardiovascular operations, chronic anemia, anemia following major surgery, coronary infarction and associated problems, chronic pulmonary disease, cardiovascular patients, autologous transfusions, as an enhancement to packed red blood cells transfusion (hemorrhage, traumatic injury, or surgery) congestive heart failure, myocardial infarction (heart attack), stroke, peripheral vascular disease, intermittent claudication, circulatory shock, hemorrhagic shock, anemia and chronic hypoxia, respiratory alkalemia, metabolic alkalosis, sickle cell anemia, reduced lung capacity caused by pneumonia, surgery, complications associated with angioplasty, pneumonia, trauma, chest puncture, gangrene, anaerobic infections, blood vessel diseases such as diabetes, substitute or complement to treatment with hyperbaric pressure chambers, intra-operative red cell salvage, cardiac inadequacy, anoxia-secondary to chronic indication, organ transplant, carbon monoxide, nitric oxide, and cyanide poisoning.

Treating a human or animal for any one or more of the above disease states may be done by transfusing into the human or animal between approximately 0.1 and 6 units (1 unit=500 mL) of blood that has been treated with a compound of the present invention. In certain cases, blood exchange with IHP-treated blood may be possible. The volume of IHP-treated red blood cells that is administered to the human or animal will depend upon the value of P₅₀ for the IHP-treated RBCs. It is to be understood that the volume of IHP-treated red blood cells that is administered to the patient can vary and still be effective. IHP-treated RBCs are similar to normal red blood cells in every respect except that their P₅₀ value is shifted towards higher partial pressures of O₂. Erythrocytes release oxygen only in response to demand by organs and tissue. Therefore, the compounds, compositions thereof, and methods of the present invention will only restore a normal level of oxygenation to healthy tissue, avoiding the cellular damage that is associated with an over-abundance of oxygen.

Because the compounds, compositions, and methods of the present invention are capable of allosterically modifying hemoglobin to favor the low oxygen affinity “T” state (i.e., right shifting the equilibrium curve), they will be useful in treating a variety of disease states in mammals, including humans, wherein tissues suffer from low oxygen tension, such as cancer and ischemia. Furthermore, as disclosed by Hirst et al. (Radiat. Res., Vol. 112, (1987), pp. 164), decreasing the oxygen affinity of hemoglobin in circulating blood has been shown to be beneficial in the radiotherapy of tumors. The compounds and compositions may be administered to patients in whom the affinity of hemoglobin for oxygen is abnormally high. For example, certain hemoglobinopathies, certain respiratory distress syndromes, e.g., respiratory distress syndromes in new born infants aggravated by high fetal hemoglobin levels, and conditions in which the availability of hemoglobin/oxygen to the tissues is decreased (e.g., in ischemic conditions such as peripheral vascular disease, coronary occlusion, cerebral vascular accidents, or tissue transplant). The compounds and compositions may also be used to inhibit platelet aggregation, antithrombotic purposes, and wound healing. Topical application could be used for wound healing.

Additionally, the compounds and compositions of the present invention can be added to whole blood or packed cells preferably at the time of storage or at the time of transfusion in order to facilitate the dissociation of oxygen from hemoglobin and improve the oxygen delivering capability of the blood. As mentioned above, when blood is stored, the hemoglobin in the blood tends to increase its affinity for oxygen by losing 2,3-diphosphoglycerides. As described above, the compounds and compositions of this invention are capable of reversing and/or preventing the functional abnormality of hemoglobin observed when whole blood or packed cells are stored. The compounds and compositions may be added to whole blood or red blood cell fractions in a closed system using an appropriate reservoir in which the compound or composition is placed prior to storage or which is present in the anticoagulating solution in the blood collecting bag. Administration to a patient can be achieved by intravenous or intraperitonal injection where the dose and the dosing regiment is varied according to individual's sensitivity and the type of disease state being treated.

Solid tumors are oxygen deficient masses. The compounds, compositions and methods of this invention may be exploited to cause more oxygen to be delivered to tumors, increasing radical formation and thereby increasing tumor killing during radiation. In this context, such IHP-treated blood will only be used in conjunction with radiotherapy.

The compounds, compositions and methods of this invention may be exploited to cause more oxygen to be delivered at low blood flow and low temperatures, providing the ability to decrease or prevent the cellular damage, e.g., myocardial or neuronal, typically associated with these conditions.

The compounds, compositions and methods of this invention may be exploited to decrease the number of red blood cells required for treating hemorrhagic shock by increasing the efficiency with which they deliver oxygen.

Damaged tissues heal faster when there is better blood flow and increased oxygen tension. Therefore, the compounds, compositions and methods of this invention may be exploited to speed wound healing. Furthermore, by increasing oxygen delivery to wounded tissue, the compounds, compositions and methods of this invention may play a role in the destruction of infection causing bacteria at a wound.

The compounds, compositions and methods of this invention will be effective in enhancing the delivery oxygen to the brain, especially before complete occlusion and reperfusion injuries occur due to free radical formation. Furthermore, the compounds, compositions and methods of this invention of this invention should reduce the expansion of arterioles under both hypoxic and hypotensive conditions.

The compounds, compositions and methods of this invention of this invention should be capable of increasing oxygen delivery to blocked arteries and surrounding muscles and tissues, thereby relieving the distress of angina attacks.

Acute respiratory disease syndrome (ARDS) is characterized by interstitial and/or alveolar edema and hemorrhage as well as perivascular lung edema associated with the hyaline membrane, proliferation of collagen fibers, and swollen epithelium with increased pinocytosis. The enhanced oxygen delivering capacity provided to RBCs by the compounds, compositions and methods of this invention can be used in the treatment and prevention of ARDS by militating against lower than normal oxygen delivery to the lungs.

There are several aspects of cardiac bypass surgery that make attractive the use of compounds or compositions or methods of the present invention. First, the compounds and compositions of the present invention act as neuroprotective agents. After cardiac bypass surgery, up to 50-70% of patients show some signs of cerebral ischemia based on tests of cognitive function. Up to 5% of these patients have evidence of stroke. Second, cardioplegia is the process of stopping the heart and protecting the heart from ischemia during heart surgery. Cardioplegia is performed by perfusing the coronary vessels with solutions of potassium chloride and bathing the heart in ice water. However, blood cardioplegia is also used. This is where potassium chloride is dissolved in blood instead of salt water. During surgery the heart is deprived of oxygen and the cold temperature helps slow down metabolism. Periodically during this process, the heart is perfused with the cardioplegia solution to wash out metabolites and reactive species. Cooling the blood increases the oxygen affinity of its hemoglobin, thus making oxygen unloading less efficient. However, treatment of blood cardioplegia with compounds or compositions of the present invention will counteract the effects of cold on oxygen affinity and make oxygen release to the ischemic myocardium more efficient, possibly improving cardiac function after the heart begins to beat again. Third, during bypass surgery the patient's blood is diluted for the process of pump prime. This hemodilution is essentially acute anemia. Because the compounds and compositions of the present invention make oxygen transport more efficient, their use during hemodilution (whether in bypass surgery or other surgeries, such as orthopedic or vascular) would enhance oxygenation of the tissues in an otherwise compromised condition. Finally, patients undergoing bypass surgery require blood transfusion after surgery. The use of compounds or compositions or methods of the present invention to make oxygen transport more efficient could obviate the need for transfusion, thus decreasing the cost of surgery and allowing the patient to avoid the risks associated with blood transfusion. Additionally, the compounds and methods of the present invention will also find use in patients undergoing angioplasty, who may experience acute ischemic insult, e.g., due to the dye(s) used in this procedure.

Additionally, microvascular insufficiency has been proposed by a number of investigators as a possible cause of diabetic neuropathy. The interest in microvascular derangement in diabetic neuropathic patients has arisen from studies suggesting that absolute or relative ischemia may exist in the nerves of diabetic subjects due to altered function of the endo- and/or epineurial blood vessels. Histopathologic studies have shown the presence of different degrees of endoneurial and epineurial microvasculopathy, mainly thickening of blood vessel wall or occlusion. A number of functional disturbances have also been demonstrated in the microvasculature of the nerves of diabetic subjects. Studies have demonstrated decreased neural blood flow, increased vascular resistance, decreased pO₂ and altered vascular permeability characteristics such as a loss of the anionic charge barrier and decreased charge selectivity. Abnormalities of cutaneous blood flow correlate with neuropathy, suggesting that there is a clinical counterpart to the microvascular insufficiency that may prove to be a simple non-invasive test of nerve fiber dysfunction. Accordingly, patients suffering from diabetic neuropathies and/or other neurodegenerative disorders will likely benefit from treatment based on the compounds and methods of the present invention.

The compounds and methods of the present invention may be used to enhance oxygen delivery in any organism, e.g., fish, that use a hemoglobin with an allosteric binding site.

II. Definitions

For convenience, certain terms employed in the specification, examples, and appended claims are collected here. As used throughout this specification and the claims, the following terms have the following meanings:

The term “hemoglobin” includes all naturally- and non-naturally-occurring hemoglobin.

The term “hemoglobin preparation” includes hemoglobin in a physiologically compatible carrier or lyophilized hemoglobin reconstituted with a physiologically compatible carrier, but does not include whole blood, red blood cells or packed red blood cells.

“Non-naturally-occurring hemoglobin” includes synthetic hemoglobin having an amino-acid sequence different from the amino-acid sequence of hemoglobin naturally existing within a cell, and chemically-modified hemoglobin. Such non-naturally-occurring mutant hemoglobin is not limited by its method of preparation, but is typically produced using one or more of several techniques known in the art, including, for example, recombinant DNA technology, transgenic DNA technology, protein synthesis, and other mutation-inducing methods.

“Chemically-modified hemoglobin” is a natural or non-natural hemoglobin molecule which is bonded to another chemical moiety. For example, a hemoglobin molecule can be bonded to pyridoxal-5′-phosphate, or other oxygen-affinity-modifying moiety to change the oxygen-binding characteristics of the hemoglobin molecule, to crosslinking agents to form crosslinked or polymerized hemoglobin, or to conjugating agents to form conjugated hemoglobin.

“Oxygen affinity” means the strength of binding of oxygen to a hemoglobin molecule. High oxygen affinity means hemoglobin does not readily release its bound oxygen molecules. The P50 is a measure of oxygen affinity.

“Cooperativity” refers to the sigmoidal oxygen-binding curve of hemoglobin, i.e., the binding of the first oxygen to one subunit within the tetrameric hemoglobin molecule enhances the binding of oxygen molecules to other unligated subunits. It is conveniently measured by the Hill coefficient (n[max]). For Hb A, n[max]=3.0.

The term “treatment” is intended to encompass also prophylaxis, therapy and cure.

“Ischemia” means a temporary or prolonged lack or reduction of oxygen supply to an organ or skeletal tissue. Ischemia can be induced when an organ is transplanted, or by conditions such as septic shock and sickle cell anemia.

“Skeletal tissue” means the substance of an organic body of a skeletal organism consisting of cells and intercellular material, including but not limited to epithelium, the connective tissues (including blood, bone and cartilage), muscle tissue, and nerve tissue.

“Ischemic insult” means damage to an organ or skeletal tissue caused by ischemia.

“Subject” means any living organism, including humans, and mammals.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitonal, transtracheal, subcutaneous, subcuticular, intraarticulare, subcapsular, subarachnoid, intraspinal and intrasternal injection and infusion.

As used herein, the term “surgery” refers to the treatment of diseases, injuries, and deformities by manual or operative methods. Common surgical procedures include, but are not limited to, abdominal, aural, bench, cardiac, cineplastic, conservative, cosmetic, cytoreductive, dental, dentofacial, general, major, minor, Moh's, open heart, organ transplantation, orthopedic, plastic, psychiatric, radical, reconstructive, sonic, stereotactic, structural, thoracic, and veterinary surgery. The method of the present invention is suitable for patients that are to undergo any type of surgery dealing with any portion of the body, including but not limited to those described above, as well as any type of any general, major, minor, or minimal invasive surgery.

“Minimally invasive surgery” involves puncture or incision of the skin, or insertion of an instrument or foreign material into the body. Non-limiting examples of minimal invasive surgery include arterial or venous catheterization, transurethral resection, endoscopy (e.g., laparoscopy, bronchoscopy, uroscopy, pharyngoscopy, cystoscopy, hysteroscopy, gastroscopy, coloscopy, colposcopy, celioscopy, sigmoidoscopy, and orthoscopy), and angioplasty (e.g., balloon angioplasty, laser angioplasty, and percutaneous transluminal angioplasty).

The term “ED₅₀” means the dose of a drug that produces 50% of its maximum response or effect. Alternatively, the dose that produces a pre-determined response in 50% of test subjects or preparations.

The term “LD₅₀” means the dose of a drug that is lethal in 50% of test subjects.

The term “therapeutic index” refers to the therapeutic index of a drug defined as LD₅₀/ED₅₀.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The term “structure-activity relationship (SAR)” refers to the way in which altering the molecular structure of drugs alters their interaction with a receptor, enzyme, etc.

The term “ammonium cation” refers to the structure below:

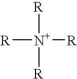

wherein R represents independently for each occurrence H or a substituted or unsubstituted aliphatic group. An “aliphatic ammonium cation” refers to the above structure when at least one R is an aliphatic group. A “quaternary ammomium cation” refers to the above structure when all four occurrences of R independently represent aliphatic groups. R can be the same for two or more occurrences, or different for all four.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aryl” as used herein includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

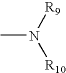

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

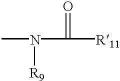

wherein R₉ is as defined above, and R′₁₁ represents a hydrogen, an alkyl, an alkenyl or —(CH₂)ₘ-R₈, where m and R₈ are as defined above.

The term “amido” is art recognized as an amino-substituted carbonyl and includes a moiety that can be represented by the general formula:

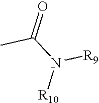

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

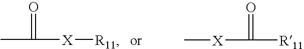

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R′₁₁ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

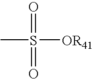

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

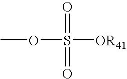

in which R₄₁ is as defined above.

The term “sulfonamido” is art recognized and includes a moiety that can be represented by the general formula:

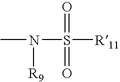

in which R₉ and R′₁₁ are as defined above.

The term “sulfamoyl” is art-recognized and includes a moiety that can be represented by the general formula:

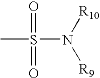

in which R₉ and R₁₀ are as defined above.

The term “sulfonyl”, as used herein, refers to a moiety that can be represented by the general formula:

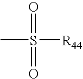

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

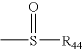

in which R₄₄ is selected from the group consisting of hydrogen, alkyl, alkenyl, alkynyl, cycloalkyl, heterocyclyl, aralkyl, or aryl.

A “phosphoryl” can in general be represented by the formula:

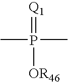

wherein Q₁ represented S or O, and R₄₆ represents hydrogen, a lower alkyl or an aryl. When used to substitute, e.g., an alkyl, the phosphoryl group of the phosphorylalkyl can be represented by the general formula:

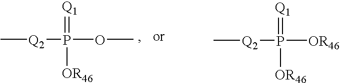

wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

III. Compounds of the Invention.

Ligands for the allosteric site of hemoglobin, also known as allosteric effectors of hemoglobin, include 2,3-diphosphoglycerate (DPG), inositol hexakisphosphate (IHP), bezafibrate (Bzf), LR16 and L35 (two recently synthesized derivatives of Bzf), and pyridoxal phosphate. Additionally, hemoglobin's affinity for oxygen can be modulated through electrostatic interactions with chloride and/or organophosphate anions present in RBCs. These effectors, which bind preferentially to the deoxy-Hb tetramers at a distance from the heme groups, play a major role in the adaptation of the respiratory properties of hemoglobin to either allometric-dependent oxygen needs or to various hypoxic environments. Additionally, protons and carbon dioxide are physiological regulators for the oxygen affinity of hemoglobin. The heterotropic allosteric interaction between the non-heme ligands and oxygen, collectively called the Bohr effect, facilitates not only the transport of oxygen but also the exchange of carbon dioxide.

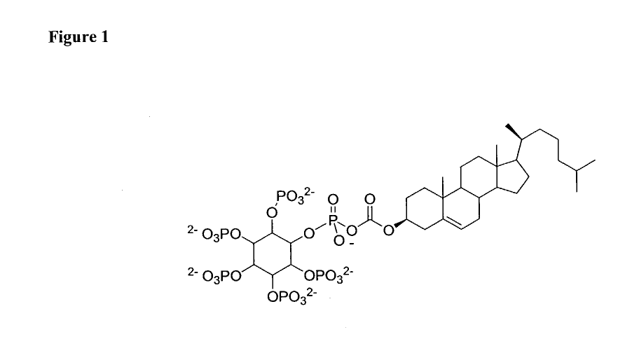

The present invention relates to compositions, and methods of use thereof, consisting essentially of a lipophilic moiety covalently bound to a ligand for the allosteric site of hemoglobin, e.g., inositol hexaphosphate (IHP). In certain embodiments, the lipophilic moiety is a sterol moiety. In certain embodiments, the sterol moiety is a cholesterol moiety (FIG. 1).

In certain embodiments, the present invention is related to compounds, and compositions thereof, which deliver into erythrocytes allosteric modifiers of hemoglobin in vivo. Additionally, the invention is directed to the use of the compounds or compositions thereof that are effective in delivering into erythrocytes allosteric modifiers of hemoglobin, lowering the oxygen affinity state in red blood cell suspensions and whole blood. It is an object of this invention to provide methods for delivering into erythrocytes allosteric modifiers of hemoglobin in whole blood and in vivo, utilizing compounds or compositions thereof that do not lose their effectiveness in the presence of normal concentrations of the remaining components of whole blood.

In part, the present invention is directed toward the design of water-soluble membrane compatible molecules comprising lipophilic moieties, e.g., lipophilic sterol moieties. These moieties are covalently bound to ligands for the allosteric site of hemoglobin; such compounds are useful for the delivery of said ligands into the cytoplasm of mammalian cells, e.g., erythrocytes.

A hydroxy group of the sterol moiety of the compounds of the present invention is particularly well suited for an oxycarbonyl attachment to an allosteric effector of hemoglobin, e.g., through a phosphate residue of IHP and congeners thereof. We report here the use of compounds comprised of a sterol moiety, i.e. cholesterol, covalently bond to ligands for the allosteric site of hemoglobin for the efficient delivery into mammalian erythrocytes of phosphate-containing ligands for the allosteric site of hemoglobin. Our data demonstrate the usefulness, convenience, and versatility of such compounds for delivery of small bioactive molecules into the cytoplasm of mammalian cells.

The covalent bond between the bioactive compound and the transport molecule must be simultaneously strong enough to remain intact before and during the transport process, and weak enough to be cleaved inside the red cell. Acyl phosphates have been shown to meet these two requirements as exemplified by several nucleoside triphosphate prodrugs studies (A. Kreimeyer, J. Ughetto-Monfrin, A. Namane, T. Huynh-Dinh Tetrahedron Lett. 1996, 37, 8739-42; D. Bonnafé, B. Dupraz, J. Ughetto-Monfrin, A. Namane, Y. Henin, T. Huynh-Dinh J. Org. Chem. 1996, 61, 895-902). For the transporter molecule, the cholesterol moiety is suitable for several reasons, including: (1) a cholesteroloxycarbonyl-ATP derivative has been shown to cross a vesicle membrane and subsequently be hydrolysed inside the vesicle (A. Kreimeyer, F. André, C. Gouyette, T. Huynh-Dinh Angew. Chem. Int. Ed. 1998, 37, 2853-55); (2) guanidinium-cholesterol derivatives have been successfully employed as vectors for gene transfer (J.-P. Vigneron, N. Oudrhiri, M. Fauquet, L. Vergely, J.-C. Bradley, M. Basseville, P. Lehn, J.-M. Lehn Proc. Nat. Acad. Sci., USA 1996, 96, 9682-86; N. Oudrhiri, J.-P. Vigneron, M. Peuchmaur, T. Leclerc, J.-M. Lehn, P. Lehn 1997, 94, 1651-56); (3) cholesterol can undergo spontaneous exchange between small unilamellar phosphatidylcholine vesicles (L. K. Bar, Y. Barenholz, T. E. Thompson Biochemistry 1986, 25, 6701-05); and (4) cholesterol can be completely exchanged from vesicle to erythrocyte membrane (B. Bloj, D. B. Zilversmit Biochemistry 1977, 18, 394347).

Importantly, as demonstrated by Kreimeyer et al., polyphosphates solubilized in organic solvents can be readily mono-acylated by reaction with one equivalent of cholesterol chloroformate. Using a similar strategy, we synthesized two IHP-cholesterol derivatives 2a and 2b, as shown in Scheme 1.

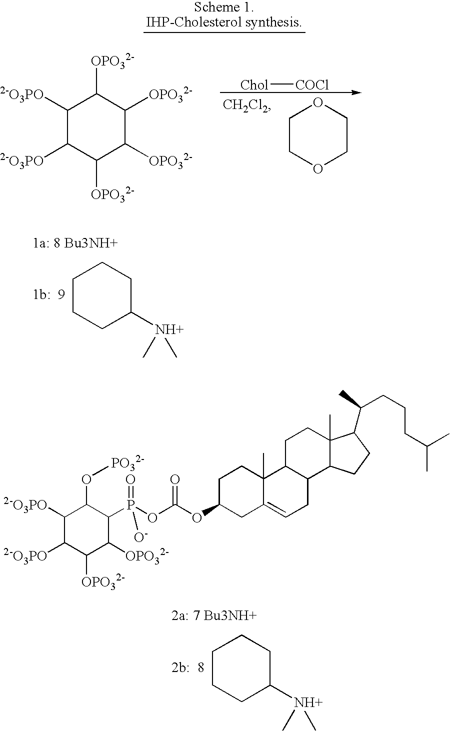

Product 2b (SV192), a preferred embodiment, was synthesized in order to perform additional biological tests. Furthermore, following the same pattern applied in the acylated derivatives we attempted a cation exchange without losing the cholesterol moiety. This transformation was found to be successful as well, (³¹P NMR), after the compound passed through a sodium cation exchange column. Scheme 2.

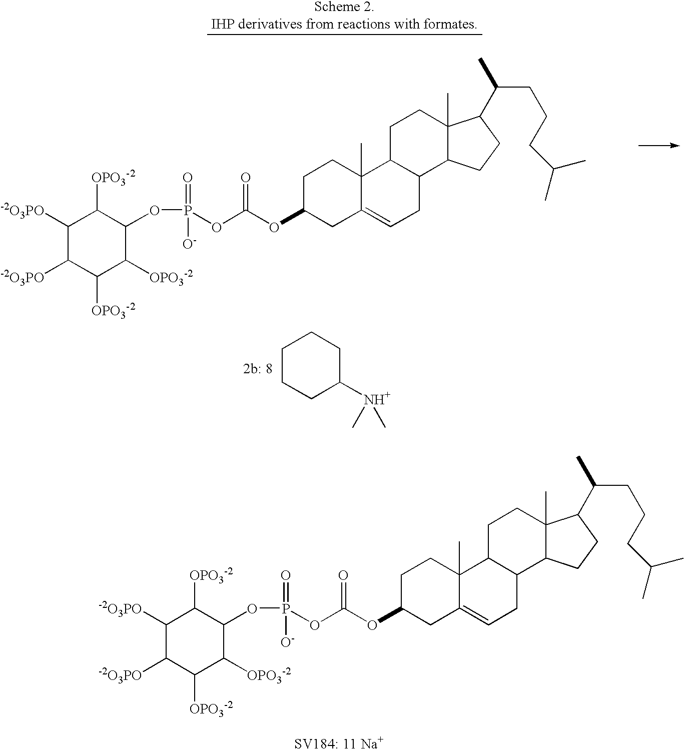

The final compounds were characterized by ¹H-, ³¹P-NMR and mass spectrometry (negative FAB). As expected, the ³¹P-NMR peaks corresponding to acylated phosphates are shifted by 10 ppm, and showed that the acylation occured non-selectively on different phosphate groups of IHP. The mass spectra showed, along with the major expected peak, two minor peaks corresponding to unreacted IHP and IHP-bis-cholesterol. HPLC chromatograms of 2a, performed on a C-18 reverse phase column, showed that around 2% of starting IHP is still present in the final crude as well as 12% of a less polar byproduct is still present. Since TLC plates showed that cholesterol or cholesterol-chloroformate are not present anymore, and given the mass spectrometry spectra, the side-product is probably the bis-acylation product of IHP.

In certain embodiments, a compound of the present invention is represented by structure 1:

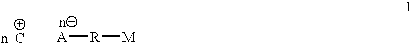

wherein

C⁺ represents independently for each occurrence an aliphatic ammonium cation, an alkali metal cation, or an alkaline earth cation; provided that at least one instance of C⁺ represents an aliphatic ammonium cation;

Aⁿ⁻ represents an anionic ligand for a mammalian cellular receptor;

R is an oxycarbonyl linking group;

M is a sterol moiety; and

n is an integer in the range 1 to 11 inclusive.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions.

In certain embodiments, the compounds of the present invention are represented by structure 1, and the attendant definitions, wherein an instance of C⁺ that represents an ammonium ion selected independently for each occurrence from the group consisting of cyclohexyl ammonium ions.

In certain embodiments, the compounds of the present invention are represented by structure 1, and the attendant definitions, wherein Aⁿ⁻ is a ligand for the allosteric site of hemoglobin.

In certain embodiments, the compounds of the present invention are represented by structure 1, and the attendant definitions, wherein Aⁿ⁻ is a phosphorylated inositol or a phosphorylated glyceric acid.

In certain embodiments, the compounds of the present invention are represented by structure 1, and the attendant definitions, wherein Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein Aⁿ⁻ is IHP or 2,3-DPG.

In certain embodiments, the compounds of the present invention are represented by structure 1, and the attendant definitions, wherein M is a cholesterol moiety.

In certain embodiments, the compounds of the present invention are represented by structure 1, and the attendant definitions, wherein R is an acyl group.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is IHP or 2,3-DPG.

In certain embodiments, the compounds of the present invention are represented by structure 1, and the attendant definitions, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is IHP or 2,3-DPG.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; M is a cholesterol moiety; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; M is a cholesterol moiety; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; M represents a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; and Aⁿ⁻ is IHP or 2,3-DPG.

In certain embodiments, the compounds of the present invention are represented by structure 1, and the attendant definitions, wherein an instance of C⁺ that represents an ammonium ion selected independently for each occurrence from the group consisting of C₃-C₆ cycloalkyl ammonium ions; M is a cholesterol moiety; and Aⁿ⁻ is IHP or 2,3-DPG.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; M is cholesterol; and Aⁿ⁻ is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; M is a cholesterol moiety; and Aⁿ⁻ is a phosphorylated inositol or a phosphorylated glyceric acid.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; and Aⁿ⁻ is IHP or 2,3-DPG.

In certain embodiments, the compounds of the present invention are represented by structure 1, and the attendant definitions, wherein an instance of C⁺ that represents an ammonium ion selected independently for each occurrence from the group consisting of cyclohexyl ammonium ions; M is a cholesterol moiety; and Aⁿ⁻ is IHP or 2,3-DPG.

In certain embodiments, the compounds of the present invention are represented by structure 1, and the attendant definitions, wherein C⁺ is a sodium ion; and Aⁿ⁻ is a phosphorylated inositol or a phosphorylated glyceric acid, wherein said phosphorylated inositol or phosphorylated glyceric acid is a ligand for the allosteric site of hemoglobin.

In certain embodiments, the compounds of the present invention are represented by structure 1, and the attendant definitions, wherein C⁺ is a sodium ion; and Aⁿ⁻ is IHP or 2,3-DPG.

In certain embodiments, the present invention relates to a pharmaceutical composition, comprising a compound of structure 1; and a pharmaceutically acceptable excipient.

In certain embodiments, a compound of structure 1 is formulated in a liposome.

In certain embodiments, a compound of structure 1 is formulated for intravenous administration.

IV. Biological Tests.

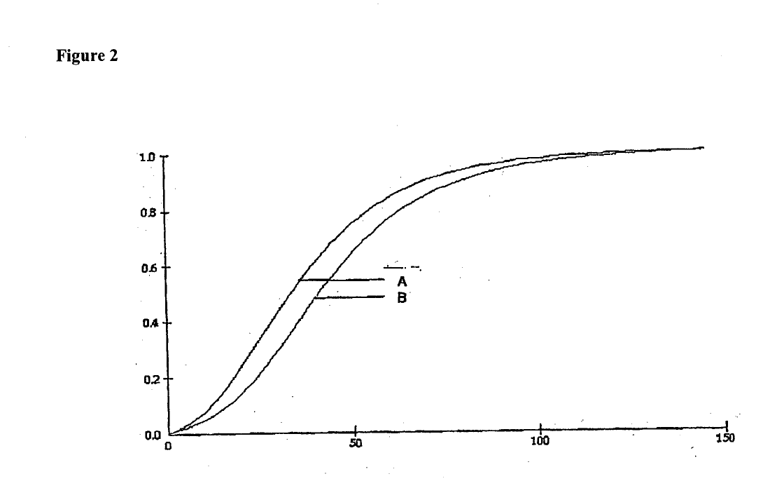

Compound 2a was synthesized and tested as its tributylammonium salt. When tested on whole blood, the IHP-Chol derivative triggered the lysis of the red blood cells. This phenomenon is attributed to the tributylammonium cations. In order to show that the cholesterol moiety did not induce hemolysis, we synthesized compound 2b, associated to N,N-dimethyl-cyclohexylammoniums, cations that did not trigger hemolysis, as shown by biological tests. As shown in Table 1, the cholesterol-IHP derivatives SV192 and SV184 displayed very interesting biological properties. See FIG. 2.

TABLE 1

| | | | pH | | |
|---|---|---|---|---|---|
| | P₅₀ | | 37° C. | | |
| | CONTROL | P₅₀ | Incubation | OSMOL. | |
| | mm Hg | mm Hg | EFF:WB | mOsM | RATIOS |
| Effector | WB | EFF:WB | EFF:fHb | EFF | EFF:WB |
| SV184 | 26 | 30 | 7.1-7.2 | 310 | 1:1.5 |
| WB | | | | | |
| SV192 | 10.5 | 32.5 | 7.1-7.2 | | 1:1(2.5 |
| fHb | | | | | mM:2.5 mM) |
| WB Standard Wash | 23,5 | | 7.1-7.2 | | 1:1.5 |
| (−C) | Lyses | no sufficient RBC | | | 1:0.375 |
| | | pellet | | | |
| WB | 25 | 29.5 | 7.1-7.2 | 224 | 1:1.5 |
| SV193 a | Non | | | | |
| | determined | | | | |
| SVI93 b | Non | | | | |
| | determined | | | | |

fHb = Free Hemoglobin ; WB = Whole Blood

To confirm these results we repeated the experiments with SV184 and SV192 obtained from different batches (SV192bis and SV192ter), varying the ratio of blood to allosteric effector, and the concentration and osmolarity of effector solutions. The solubility of each compound was tested in water (pH adjusted to 7.1-7.2 and osmolarity adjusted with glucose to 300-330 mOsM.) SV192 was difficult to dissolve giving a turbid solution after 10 minutes of sonication, SV192bis was easy to dissolve giving a clear solution without sonication, and SV192ter gave a milky solution after strong sonication.

Effector and whole blood (WB) at 1:0.375 and 1:1.5 ratios were incubated at 37° C. for 10-60 minutes. Following incubation, blood cells were washed three times with 20 mM Bis-Tris buffer. P50 of effector incubated with RBC were recorded using a Hemox Analyzer and a HAS software from TCS Scientific (New Hope, Pa.). The reading solution consisted of an aliquot of RBCs to meet instrument parameters and 20 mM Hemox buffer (Osm=310±10 mOsM, pH=7.1-7.2) at 37° C. The results of the P50 measurements are shown in Table 2.

TABLE 2
P50 values of human and mouse RBCs in whole blood after incubation
with monocholesterol-IHP in vitro.

| | P50 mm | | | | | |
|---|---|---|---|---|---|---|
| | Hg | P50 mm | P50 | Incubation | Osmol. | |
| Effector | Control | HG | Increase | Ratio | mOsM | |
| Name | WB | EFF:WB | % | EFF:WB | EFF | Conc. |
| SV184 | | | | | | |
| Human | 26.2 | 30.4 | 16 | 1:1.5 | 232 | 30 mM |
| Mouse | 38.3 | 41.8 | 9 | 1:1.5 | 310 | 30 mM |
| | | 44.3 | 15 | 1:0.375 | 224 | 30 mM |
| SV192 | | | | | | |
| Human | 23.5 | 29.5 | 26 | 1:1.5 | 224 | 30 mM |
| | 25 | lysis | — | 1:0.375 | 224 | 30 mM |
| | 24 | 30 | 25 | 1:1.5 | 310 | 30 mM |
| SV192bis | | | | | | |
| Human | 25 | 26.6 | 6 | 1:1.5 | 220 | 30 mM |
| Human | | 24 | 6 | 1:1.5 | 272 | 30 mM |
| Human | | 28.3 | 13 | 1:1.5 | 306 | 30 mM |
| SV192ter | | | | | | |
| Mouse | 38.3 | 41.4 | 8 | 1:1.5 | 320 | 30 mM |
| | | 43.4 | 13 | 1:1.5 | 320 | 30 mM |
| | | 41.9 | 9 | 1:1.5 | 320 | 30 mM |
| Mouse | 40.4 | 42.6 | 5 | 1:1.5 | 320 | 50 mM |
| | | 43.1 | 7 | 1:1.5 | 283 | 5 mM |
| Mouse | 41.3 | 42.2 | 2 | 1:1.5 | 264 | 15 mM |
| | | 43.5 | 5 | 1:1.5 | 276 | 30 mM |
| | | 43.1 | 4 | 1:1.5 | 314 | 60 mM |
| Mouse | 41.3 | 46.4 | 8 | 1:0.375 | 220 | 30 mM |
| Human | 26.1 | 26.8 | 3 | 1:1.5 | 274 | 30 mM |
| Human | 25.0 | 26.3 | 5 | 1:1.5 | 202 | 30 mM |
| | | 25.6 | 2 | 1:1.5 | 258 | 30 mM |
| | | 26.2 | 4 | 1:1.5 | 303 | 30 mM |
| Human | 26.1 | 26.8 | 3 | 1:1.5 | 274 | 30 mM |
| Human | 24.3 | 26.3 | 5 | 1:1.5 | 202 | 30 mM |
| | | 25.6 | 2 | 1:1.5 | 258 | 30 mM |
| | | 26.2 | 4 | 1:1.5 | 303 | 30 mM |

SV192 gave a significant shift in vitro, both at relatively low and at nearly physiological osmolarity. Even at nearly physiological osmolarity and at a volume ratio of effector solution:whole blood (WB)=1:1.5, the shift is 25%, which makes SV192 a good candidate for in vivo work. The lysis induced by SV192 solution in very low volumes of whole blood was due to low osmolarity combined with the effector's action on the RBC membranes. SV192bis and SV192ter also gave at nearly physiological osmolarity significant (>10%) shifts of the P50 value both with human and with mouse blood. The volume ratio effector solution:whole blood was 1:1.5 and the concentration of effector was 30 mM. However, SV192ter gave such a shift with mouse blood only, whereas 192bis gave it with human blood. SV192bis gave the expected ³¹P NMR spectrum, showing the cholesterol moiety and the IHP molecule. Next, we looked into the dependence of the P50 value on the effector concentration. The effector was SV192ter and the results are presented in Table 3.

TABLE 3
Dependence of the P50 value on the effector concentration in the incubation medium.

| | P50 mm | | | | | | |
|---|---|---|---|---|---|---|---|
| | Hg | P50 mm | P50 | Incubation | Osmol. | pH/37° C. | |
| | Control | Hg | Increase | Ratio | mOsM | Hemox | |
| Effector | WB | Eff:WB | % | EFF:WB | EFF | Chamber | Conc. |
| SV192ter | | | | | | | |
| Mouse | 40.39 | 42.6 | 5 | 1:1.5 | 320 | 7.2-7.4 | 50 mM |
| Mouse | | 43.1 | 7 | 1:1.5 | 283 | 7.2-7.4 | 5 mM |
| Mouse | 41.3 | 42.2 | 2 | 1:1.5 | 264 | 7.2-7.4 | 15 mM |
| Mouse | | 43.5 | 5 | 1:1.5 | 276 | 7.2-7.4 | 30 mM |
| | | 43.1 | 4 | 1:1.5 | 314 | 7.2-7.4 | 60 mM |

Increased concentrations of the effector did not produce higher P50 values in vitro. Apparently, the concentration of 30 mM already induces the maximum shift obtainable with the mono cholesterol-IHP compound. P50 shifts below 5% of the control value are not physiologically significant, since the lowest shift of the P50 of all the circulating blood known to induce physiological effects in vivo is approximately 7% of the control value.

The in vitro work indicated that the monocholesterol derivative was capable of crossing the plasma membrane of the red blood cells and induced a right shift of the HbO₂ dissociation curve. Increases of the P50 values of the red blood cells from the whole blood incubated with these derivatives reached up to 26% of the control value with human blood and up to 13% with mouse blood. At physiological or nearly physiological osmotic pressure no lysis of the RBC was induced by SV192, SV192bis, or SV192ter. Effector concentrations beyond 30 nM do not increase the P50 shift further.

The tolerability and pharmacodynamics of the compounds SV184 and SV192 were tested in a small number of C57B1/6 mice. Mice receiving the whole volume of 200 μL of (30 mM) effector solution died immediately after injection or during injection (injection time 10-20 min). Eight mice receiving less than 200 μL of the compound solution survived. These mice were fast breathing and shaking, but recovered after 4 hours. Blood from these animals (taken 4 and 24 hours after injection) did not show a significant shift of P50. Table 4 shows the results. The mono-cholesterol IHP derivatives SV184 and SV192 showed, upon iv injection a high level of toxicity in mice. Therefore, new derivatives were synthesized in our labs.

TABLE 4
Administration of SV184 and SV192 to C5B1/6 mice.

| | Number of | Number of | Shift of | |
|---|---|---|---|---|
| Effector Name | injected mice | mice died | P50% | Injection |
| SV192 | 1 | 1 | — | 200 μL of 30 mM |
| | 2 | 2 | — | 165 μL of 30 nM |
| | 2 | 0 | <3 | 135 μL of 30 nM |
| SV184 | 1 | 1 | — | 200 μL of 30 nM |
| | 4 | 0 | <5 | 150 μL of 30 nM |

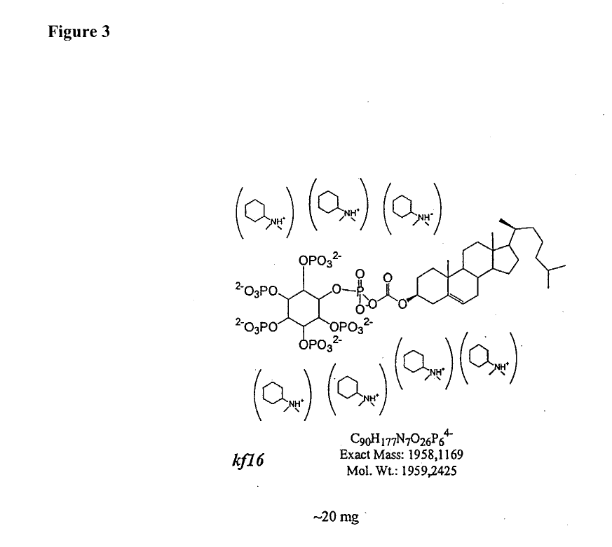

The three inositol hexaphosphate cholesterol derivatives KF16, FK16a3, and KF16.3 were synthesized and purified by different procedures. See FIG. 3 and Table 9. They were soluble in water and tested for P50 right shift with both mouse hemoglobin and whole blood. The pH of the compound solutions was adjusted to approximately 7.1 and effector solution (2.5 mM) and mouse hemoglobin (2.5 mM) were mixed at 1:1 ratio. The osmolarity was adjusted to 218-310 mOsM. Effectors and whole blood (WB) at 1:1.5 were incubated at 37° C. for 60 minutes. Following incubation, blood cells were washed three times with 20 mM Bis-Tris buffer. A summary of P50 values of free hemoglobin induced by the newly synthesized KF-effectors is presented in Table 5.

TABLE 5
P50 values of C57B1/6 mice free hemoglobin after incubation with
compounds KF16, FK16a3, and KY16.3 in vitro.

| | | | | pH/20° C. |
|---|---|---|---|---|
| | P50 mm Hg | P50 mm Hg | P50 | Compound |
| Effector Name | Control fHb | EFF/fHb | Increase % | Solution |
| KF16 | 27.43 | 52.17 | 90 | 7.0 |
| KF16.3 | 12.34 | 32.46 | 163* | 7.0 |
| KF16a3 | 26.05 | 31.54 | 21 | 7.0 |

*concentration of effector was 30 mM

These compounds induced a far stronger right shift of the P50 value of mouse hemoglobin than the SV-derivatives. Compound-solutions adjusted to physiological osmolarity (280-310) did not show a significant right shift of P50 in whole blood at concentrations of 30 mM. There was some lysis induced by all compounds due to low osmolarity combined with the effectors' action on the RBC membranes. These effectors were then tested for P50 shifts in mouse whole blood. Again, pH of the compound solutions was adjusted and effectors and whole blood at 1:1.5 volume ratios were incubated. Following incubation, blood cells were washed as usual. A summary of P50 values in whole blood induced by the effectors is presented in Table 6. The compound KF16.3 at lower osmolarity induced a strong right shift of the P50 value (more than 30%) with whole blood, making it a good candidate for further investigation.

TABLE 6
P50 values in mouse
whole blood after incubation with KF-compounds in vitro.

| | | | | | pH/ | |
|---|---|---|---|---|---|---|
| | | | | | 20° C. | |
| | P50 mm | | | | Com- | |
| | Hg | P50 mm | P50 | Osmol. | pound | |
| Effector | Control | Hg | Increase | mOsM | Solu- | |
| Name | WB | EFF:WB | % | EFF | tion | Conc. |
| KF16* | 37.29 | 36.14 | — | 283 | 7.1 | 30 mM |
| KF16a3* | 33.80 | 39.70 | 17 | 219 | 7.1 | <30 mM |
| KF16.3* | 37.45 | 54.60 | 46 | 218 | 7.0 | <30 mM |

*KF16 obtained from different syntheses.

Based on this result, a preliminary investigation of the effects of intravenous (iv) injection of the compound KF16.3 was undertaken. Two routes of in vivo administration of the compound, KF16.3, were undertaken.

Route 1

Blood (100-120 μL) was collected from the tail vein of a C57B1/6 mouse. About 50-70 μL of the blood was taken for control measurements. The rest of the blood was incubated for 60 minutes with the same volume of a freshly prepared KF16.3 solution (approximately 30 mM, pH 7.1, 218 mOsM). Following incubation blood cells were washed as described. Some lysis occurred. The remaining intact RBCs (volume of 30-45 μL) were reinjected into the mouse's tail vein and 30-45 minutes later blood was taken again in order to measure P50 shift. The reinjection of the incubated blood was tolerated well by the mice. A significant right shift of P50 was observed in two animals injected. The results are shown in Table 7.

TABLE 7
P50 values of mouse blood 30-45 minutes after reinjection
of autologous KF16.3 preincubated blood.

| | | P50 mm Hg | | | |
|---|---|---|---|---|---|
| | | Mouse RBCs | P50 | P50 mm Hg | P50 |
| Effector | P50 mm Hg | loaded ex | Increase | Circulating | Increase |
| Name | Control | vivo | % | Blood | % |
| KF16.3 | 47.9 | 69.9 | 46 | 54.0 | 13 |
| | 37.4 | — | — | 42 | 12.2 |

Route 2

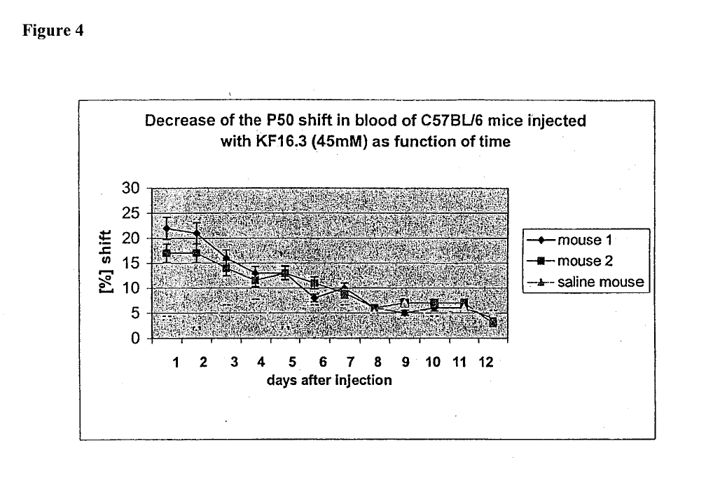

In order to evaluate the tolerability of the compound by the mice, we administered KF16.3 at different concentrations (30, 45 and 60 mM). The pH was adjusted to 7.1-7.2, and 200 μL of the solutions were administered via iv to 4 C57B1/6 mice. Mice were observed for 2 hours. The substance was tolerated well in all concentrations. No mouse died. Mice recovered quickly after ether-anesthesia and did not show any differences in their behavior to mice receiving the same volume of saline. Two hours after injection blood samples were taken for P50 measurements. The P50 values are summarized in Table 8. Blood from mice receiving 200 μL of the 30 mM solution showed a right shift of 8-11%. P50 shifts in blood from mice receiving 45 mM of the effector solution were measured over 12 days. The results are shown in FIG. 4. Following injection of 45 mM KF16.3 there was a strong right shift of P50 in both animals (17 and 22%). In observation during 12 days (half life of mouse RBC) the treated animals show a progressive decrease in P50. This decrease correlates with the in vivo half life of the erythocytes.

TABLE 8
P50 values of mouse whole blood after in vivo injection of
compound KF16.3 at different concentrations.

| | P50 mm Hg | P50 mm Hg | | |
|---|---|---|---|---|
| | Control before | 2 h after | | |
| Effector Name | injection | injection | P50 Increase % | Conc. |
| KF16.3 | 48.3 | 52.56 | 8.8 | 30 mM |
| | 45.44 | 47.47 | 6.7 | 45 mM |
| | 39.41 | 43.44 | 10.2 | 60 mM |
| | 47.79 | 54.86 | 14.8 | 60 mM |

Based on these results, the following conclusions were made: 1) KF16.3 was shown to cause a right shift in the P50 value of both free hemoglobin and whole blood, the percentage of P50 increase in whole blood was 46%; 2) in vivo administration of different concentrations of KF16.3 showed a right shift of the whole circulating blood of the injected mice, shift being between 7-15% (demonstrated on 4 animals); 3) administration of the KF compounds by reinjection of autologous blood, incubated with KF16.3 showed a significant right shift in the P50 value of circulating blood taken 30-45 minutes after injection (shift of 13%, demonstrated on 2 animals); and 4) the tolerability of the compound was tested by direct injection into the vein at 30, 45 and 60 mM: KF16 was non-toxic at these concentrations, and a significant shift of P50 value (>10%) was achieved after injection of 200 μL of a 60 mM compound-solution. Lower concentrations did cause a right shift in P50 value of 6-9% when injected. However, the number of animals used (1−2 animals per concentration) was low.

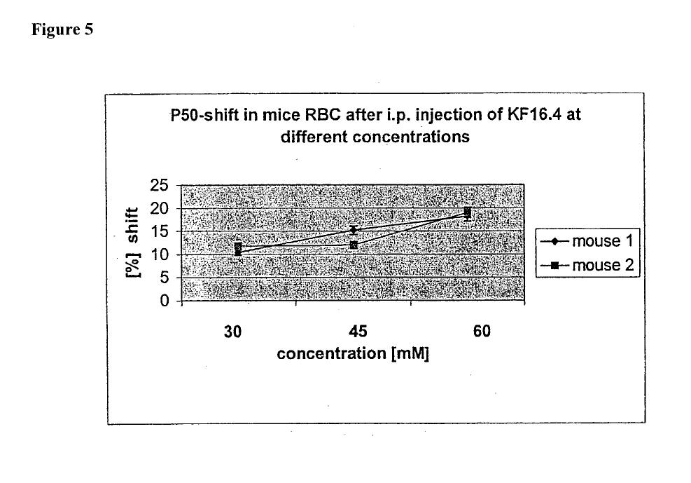

KF16 was also administered at 3 concentrations (30, 45 and 60 mM) by intraperitonal (ip) injection. The substance was tolerated well in all concentrations; no mouse died. Three hours after injection, samples of blood were taken for P50 measurements. There was a strong right shift of P50 observed in each animal injected. We have observed the shift of the P50 values in the blood from the injected animals over 3 days. The results are shown in FIG. 5.

Intraperitonal injections are well tolerated by the mice even at concentrations of 60 mM. The P50 shift of RBC is similar to iv injections at concentrations of 30 and 45 mM. After ip injections with KF16.4 there is a shift of 12 to 18% following the increasing concentration of the effector (30-60 mM). Two important facts emerge from these measurements: 1) a reproducible right shift in the Hb-O₂ dissociation curve is observed in vivo upon iv or ip injection of the KF16 compounds, this shift is concentration dependent; and 2) the decrease of the P50 shift over time is progressive and correlates with the life-time of the mouse RBC, this indicates that we have a true shift, induced by the allosteric effector injected and not an artifact.

It became apparent that the same structural effector could have different properties depending upon how it was prepared and purified. Compounds KF16, KF16.3 and KF16a3 along with compounds KF92p, KF93p, KF16.4, and KF16.5 were prepared and purified by the methods in Table 9.

TABLE 9
Methods and purification conditions for KF16, KF16.3,
KF16a3, KF16.4, KF16.5, KF92p, and KF93p.

| Effector | | |
|---|---|---|
| Name | Reaction Conditions | Purification Conditions |
| KF16 | 1 eq CholOCOCl | Extraction |
| | CH₂Cl₂/1,4-dioxane 2.5/L | CH₂Cl₂/H₂O |
| | (0.017 M to IHP salt) | |
| KF16a3 | 1 eq CholOCOCl | Extraction |
| | CH₂Cl₂/1,4-dioxane 2.5/L | Hexanes/MeOH heat to 50° C., |
| | (0.017 M to IHP salt) | cool and separate |
| KF16.3 | 1 eq CholOCOCl | Wash with hexanes, |
| | CH₂Cl₂/1,4-dioxane 2.5/L | hexanes/CH₂Cl₂ |
| | (0.017 M to IHP salt) | |
| KF16.4 | 1 eq CholOCOCl | Wash with hexanes, |
| | CH₂Cl₂/1,4-dioxane 2.5/L | hexanes/CH₂Cl₂ 9/1 |
| | (0.017 M to IHP salt) | |
| KF16.5 | 1 eq CholOCOCl | Wash with hexanes, |
| | CH₂Cl₂/1,4-dioxane 2.5/L | hexanes/CH₂Cl₂ 9/1 |
| | (0.017 M to IHP salt) | centrifugation |
| KF92p | 1.5 eq Chol | Wash with hexanes, |
| | THF:CH₃CN 1:1 | Hexanes/THF 9/1 |
| | 22 hours | centrifugation |
| | (0.024 M to IHP) | |
| KF93p | 2 eq Chol | Wash with hexanes, |
| | THF:CH₃CN 1:1 | Hexanes/THF 9/1 |
| | 22 hours | centrifugation |
| | (0.024 M to IHP) | |

The compounds KF16.3, KF16.4, KF16.5, KF92p, and KF93p are soluble in water. They were tested for P50 right shift with mouse hemoglobin. The pH was adjusted to approximately 7.0 and effector solution (2.5 mM) and mouse hemoglobin (2.5 mM) were mixed at 1:1 ratio. P50 values were measured as before. A summary of P50 values of free hemoglobin induced by the KF16 effectors is presented in Table 10.

TABLE 10
P50 values of C57B1/6 mice free hemoglobin after incubation with
compounds KF16 in vitro.

| | | | | pH/37° C. |
|---|---|---|---|---|
| | P50 mm Hg | P50 mm Hg | | Hemox |
| Effector Name | Control WB | EFF:WB | P50 Increase % | Chamber |
| KF92p | 18.0 | 48.8 | 171 | 7.0 |
| KF93p | 19.9 | 46.8 | 135 | 7.0 |
| KF16.3 | 17.0 | 53.9 | 200 | 7.0 |
| KF16.4 | 15.5 | 43.6 | 180 | 7.0 |
| | 17.0 | 56.5 | 156 | 7.0 |
| KF16.5 | 18.0 | 58.2 | 223 | 7.0 |

Each batch of compound KF16 induced a strong right shift of the P50 value of mouse hemoglobin (150-225%). They were then tested for P50 shifts in mouse whole blood. Again, pH of the compound solutions was adjusted and effectors and whole blood at 1:1.5 volume ratios were incubated. Following incubation, blood cells were washed as usual. A summary of P50 values in whole blood induced by the effector obtained from different syntheses is presented in Table 11.

TABLE 11
P50 values in mouse RBC after incubation with KF-compounds in vitro.

| | P50 | | | | | | |
|---|---|---|---|---|---|---|---|
| | mm Hg | P50 mm | P50 | Incubation | Osmol. | pH/37° C. | |
| Effector | Control | Hg | Increase | Ratio | mOsM | Hemox | |
| Name | WB | EFF:WB | % | EFF:WB | EFF | Chamber | Conc. |
| KF92p | 40.6 | 43.1 | 6 | 1:1.5 | 217 | 7.0 | ˜30 |
| | | | | | | | mM |
| KF93p | 38.8 | 43.0 | 11 | 1:1.5 | 214 | 7.0 | 30 mM |
| KF16.3 | 36.6 | 47.9 | 31 | 1:1.5 | 220 | 7.0 | 30 mM |
| KF16.4 | 36.0 | 46.8 | 30 | 1:1.5 | 217 | 7.0 | 30 mM |
| | 34.6 | 42.9 | 24 | 1:1.5 | 220 | 7.0 | 30 mM |
| | 35.7 | 41.8 | 17 | 1:1.5 | 296 | 7.0 | 30 mM |
| | 35.7 | 52.8 | 48 | 1:1.5 | 221 | 7.0 | 30 mM |
| KF16.5 | 36.6 | 48.7 | 31 | 1:1.5 | 221 | 7.0 | 30 mM |

Compounds KF16.3, KF16.4 and KF16.5 at lower osmolarity (approximately 220 mOsM) induced a strong right shift of the P50 value (25-48%) in whole blood; KF16.4 also gave a right shift of 17% at isoosmolar condition. KF16.3, KF16.4, KF16.5, KF92p and KF93p cause a right shift in the P50 value of both free hemoglobin and whole blood. The percentage of P50 increase in hemoglobin was up to 225%, and in whole blood it was up to 48%. In vivo administration of 200 μL of a 45 mM KF16.3 solution showed a right shift of the whole circulating blood of the injected mice, shifts being up to 22% (demonstrated on 2 animals). Administration of the KF16.4 compound intraperitonally showed a significant right shift in the P50 of up to 18% (demonstrated on 6 animals). The tolerability of the compound was tested by direct injection into the vein at 30 and 45 mM. KF16 was non-toxic at these concentrations. The tolerability of the compound was tested also by intraperitonal injection at 30, 45 and 60 mM. KF 16 was non toxic at these concentrations. Ex vivo, fluorescence data suggest that approximately 11% of the RBC take up the KF 16 compound.

IV. Methods of the Invention

In certain embodiments, the method of the present invention comprises the step of administering to a subject a therapeutically effective amount of a compound or composition of the present invention.

In certain embodiments, the method of the present invention comprises the step of administering to a subject a therapeutically effective amount of a compound or composition of the present invention, wherein said administration is intravenous.

In certain embodiments, the method of the present invention comprises the step of administering to a subject experiencing ischemia a therapeutically effective amount of a compound or composition of the present invention.

In certain embodiments, the method of the present invention comprises the step of administering to a subject experiencing ischemia a therapeutically effective amount of a compound or composition of the present invention, wherein said administration is intravenous.

In certain embodiments, the method of the present invention comprises the step of administering to a subject experiencing cardiac arrhythmia a therapeutically effective amount of a compound or composition of the present invention.

In certain embodiments, the method of the present invention comprises the step of administering to a subject experiencing cardiac arrhythmia a therapeutically effective amount of a compound or composition of the present invention, wherein said administration is intravenous.

In certain embodiments, the method of the present invention comprises the step of administering to a subject experiencing a heart attack a therapeutically effective amount of a compound or composition of the present invention.

In certain embodiments, the method of the present invention comprises the step of administering to a subject experiencing a heart attack a therapeutically effective amount of a compound or composition of the present invention, wherein said administration is intravenous.

In certain embodiments, the method of the present invention comprises the step of administering to a subject experiencing a stroke a therapeutically effective amount of a compound or composition of the present invention.

In certain embodiments, the method of the present invention comprises the step of administering to a subject experiencing a stroke a therapeutically effective amount of a compound or composition of the present invention, wherein said administration is intravenous.

In certain embodiments, the method of the present invention comprises the step of administering to a subject experiencing hypoxia a therapeutically effective amount of a compound or composition of the present invention.

In certain embodiments, the method of the present invention comprises the step of administering to a subject experiencing hypoxia a therapeutically effective amount of a compound or composition of the present invention, wherein said administration is intravenous.

In certain embodiments, the method of the present invention comprises the step of administering to a subject afflicted with sickle cell anemia a therapeutically effective amount of a compound or composition of the present invention.

In certain embodiments, the method of the present invention comprises the step of administering to a subject afflicted with sickle cell anemia a therapeutically effective amount of a compound or composition of the present invention, wherein said administration is intravenous.

In certain embodiments, the method of the present invention comprises the step of administering to a subject suffering from hypotension a therapeutically effective amount of a compound or composition of the present invention.

In certain embodiments, the method of the present invention comprises the step of administering to a subject suffering from hypotension a therapeutically effective amount of a compound or composition of the present invention, wherein said administration is intravenous.

In certain embodiments, the method of the present invention comprises the step of administering to a subject suffering from arteriosclerosis a therapeutically effective amount of a compound or composition of the present invention.

In certain embodiments, the method of the present invention comprises the step of administering to a subject suffering from arteriosclerosis a therapeutically effective amount of a compound or composition of the present invention, wherein said administration is intravenous.

In certain embodiments, the method of the present invention comprises the step of administering to a subject suffering from altitude sickness a therapeutically effective amount of a compound or composition of the present invention.

In certain embodiments, the method of the present invention comprises the step of administering to a subject suffering from altitude sickness a therapeutically effective amount of a compound or composition of the present invention, wherein said administration is intravenous.

In certain embodiments, the method of the present invention comprises the step of administering to a subject suffering from diabetes a therapeutically effective amount of a compound or composition of the present invention.

In certain embodiments, the method of the present invention comprises the step of administering to a subject suffering from diabetes a therapeutically effective amount of a compound or composition of the present invention, wherein said administration is intravenous.

In certain embodiments, the method of the present invention comprises the step of adding to mammalian blood a compound or composition of the present invention.

In certain embodiments, the method of the present invention comprises the step of adding to plasma comprising mammalian erythrocytes a compound or composition of the present invention.

V. Specific Clinical Applications of Enhanced Oxygen Delivery

There are numerous clinical conditions that would benefit from treatments that would increase tissue delivery of oxygen bound to hemoglobin. For example, the leading cause of death in the United States today is cardiovascular disease. The acute symptoms and pathology of many cardiovascular diseases, including congestive heart failure, myocardial infarction, stroke, intermittent claudication, and sickle cell anemia, result from an insufficient supply of oxygen in fluids that bathe the tissues. Likewise, the acute loss of blood following hemorrhage, traumatic injury, or surgery results in decreased oxygen supply to vital organs. Without oxygen, tissues at sites distal to the heart, and even the heart itself, cannot produce enough energy to sustain their normal functions. The result of oxygen deprivation is tissue death and organ failure.

Although the attention of the American public has long been focused on the preventive measures required to alleviate heart disease, such as exercise, appropriate dietary habits, and moderation in alcohol consumption, deaths continue to occur at an alarming rate. Since death results from oxygen deprivation, which in turn results in tissue destruction and/or organ dysfunction, one approach to alleviate the life-threatening consequences of cardiovascular disease is to increase oxygenation of tissues during acute stress. The same approach is also appropriate for persons suffering from blood loss or chronic hypoxic disorders, such as congestive heart failure.

Another condition which could benefit from an increase in the delivery of oxygen to the tissues is anemia. A significant portion of hospital patients experience anemia or a low “crit” caused by an insufficient quantity of red blood cells or hemoglobin in their blood. This leads to inadequate oxygenation of their tissues and subsequent complications. Typically, a physician can temporarily correct this condition by transfusing the patient with units of packed red blood cells.

Enhanced blood oxygenation may also reduce the number of heterologous transfusions and allow use of autologous transfusions in more case. The current method for treatment of anemia or replacement of blood loss is transfusion of whole human blood. It is estimated that three to four million patients receive transfusions in the U.S. each year for surgical or medical needs. In situations where there is more time it is advantageous to completely avoid the use of donor or heterologous blood and instead use autologous blood.

Often the amount of blood which can be drawn and stored prior to surgery limits the use of autologous blood. Typically, a surgical patient does not have enough time to donate a sufficient quantity of blood prior to surgery. A surgeon would like to have several units of blood available. As each unit requires a period of several weeks between donations and can not be done less than two weeks prior to surgery, it is often impossible to sequester an adequate supply of blood. By processing autologous blood with IHP, less blood is required and it becomes possible to completely avoid the transfusion of heterologous blood.

Because IHP-treated RBCs may release up to 2-3 times as much oxygen as untreated red cells, in many cases, a physician will need to transfuse fewer units of IHP-treaded red cells. This exposes the patient to less heterologous blood, decreases the extent of exposure to vital diseases from blood donors and minimizes immune function disturbances secondary to transfusions. The ability to infuse more efficient red blood cells is also advantageous when the patients blood volume is excessive. In more severe cases, where oxygen transport is failing, the ability to improve rapidly a patient's tissue oxygenation is life saving.

Although it is evident that methods of enhancing oxygen delivery to tissues have potential medical applications, currently there are no methods clinically available for increasing tissue delivery of oxygen bound to hemoglobin. Transient, 6 to 12 hour elevations of oxygen deposition have been described in experimental animals using either DPG or molecules that are precursors of DPG. The natural regulation of DPG synthesis in vivo and its relatively short biological half-life, however, limit the DPG concentration and the duration of increased tissue PO₂, and thus limit its therapeutic usefulness.

Additionally, as reported in Genetic Engineering News, Vol. 12, No. 6, Apr. 15, 1992, several groups are attempting to engineer free oxygen-carrying hemoglobin as a replacement for human blood. Recombinant, genetically modified human hemoglobin that does not break down in the body and that can readily release up to 30% of its bound oxygen is currently being tested by Somatogen, Inc., of Boulder Colo. While this product could be useful as a replacement for blood lost in traumatic injury or surgery, it would not be effective to increase PO₂ levels in ischemic tissue, since its oxygen release capacity is equivalent to that of natural hemoglobin (27-30%). As are all recombinant products, this synthetic hemoglobin is also likely to be a costly therapeutic.

Synthetic human hemoglobin has also been produced in neonatal pigs by injection of human genes that control hemoglobin production. This product may be less expensive product than the Somatogen synthetic hemoglobin, but it does not solve problems with oxygen affinity and breakdown of hemoglobin in the body.

VI. Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present compounds contain a cationic or basic functional group, such as an ammonium ion, or amino or alkylamino group, and are, thus, capable of forming pharmaceutically-acceptable salts with pharmaceutically-acceptable acids, e.g., a ligand for the allosteric site of hemoglobin such as inositol hexaphosphate (IHP). These salts can be prepared in situ during the final isolation and purification of the compounds of the invention, or by separately reacting a first salt of the invention with a second suitable organic or inorganic salt, and isolating the new salt thus formed. For example, the chloride salt of a guanidinylated sterol may be combined with the potassium salt of IHP to give potassium chloride and a salt of the present invention comprising the guanidinylated sterol and IHP. In this regard, representative anions include bromide, chloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate; and representative cations include sodium, potassium, lithium, cesium, magnesium, calcium, and barium.

In certain cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include, but are not limited to, those suitable for parenteral administration. The formulations may conveniently be presented in unit dosage form and may be prepared by any methods well known in the art of pharmacy. For example, the amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, or phenol sorbic acid. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

In some cases, in order to prolong the effect of a drug, it is desirable to slow the absorption of the drug, e.g., from a subcutaneous or intramuscular injection. This goal may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers, such as polylactide-polyglycolide. In certain embodiments, the monomers of the biodegradable polymer comprise the functionalized sterol of the salts of the present invention, so that as the polymer biodegrades the salt of the present invention is released over a desired period of time. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Intravenous administrations are preferred.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intravectally, for example, as a pessary, cream or foam.

The compounds according to the invention may be formulated for administration in any convenient way for use in human or veterinary medicine, by analogy with other pharmaceuticals.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

VII. Administration of the Compounds of the Present Invention

Many techniques currently exist for delivering drugs or other medicaments to body tissue. These include, among possible others, oral administration, injection directly into body tissue such as through an intramuscular injection or the like, topical or transcutaneous administration where the drug is passively absorbed, or caused to pass, into or across the skin or other surface tissue and intravenous administration which involves introducing a selected drug directly into the blood stream.

Except for topical or transcutaneous administration, the above drug delivery systems tend to be systemic. In other words, administration of the drug is delivered throughout the body by the blood stream.

Several devices have been developed for the purpose of accessing specific body lumens or passageways (i.e., blood vessels, gastrointestinal tract, urinary tract) and delivering therapeutic agents transmurally to specific subregions of tissue. A double-balloon catheter has been used to administer agents to the area confined by the balloons. A disadvantage of this system is that drugs may be lost through communicating vessels between the balloons. Alternatively, a perforated balloon has been developed to deliver agents directly into the vessel wall. A major disadvantage with both of these systems in certain desired applications is that the drug is delivered radially in all directions.

It is contemplated that other transport forces could also be used either with or in lieu of pressure to enhance or otherwise control the speed of drug transport. For example, one method could utilize DMSO as a carrier to transport a fixative or drug through the vessel wall. Other fluid diffusion enhancement compositions include propylene glycol, azone and ionic or non-ionic surfactants.

The parenteral administration of medical liquids is an established clinical practice. The liquids are administered particularly intravenously, and the practice is used extensively as an integral part of the daily treatment of medical and surgical patients. The liquids commonly administered include blood and blood substitutes, dextrose solution, electrolyte solution, and saline. Generally, the liquids are administered from an intravenous delivery system having a container suspended above the patient, with the liquid flowing through a catheter hypodermic needle set to the patient.

The administration of liquids intravenously is a valuable and important component that contributes to the optimal care of the patient; however, it does not always provide a satisfactory means and method for administering concomitantly therewith a beneficial agent. Presently, a beneficial agent is often administered intravenously by (1) temporarily removing the intravenous system and halting the flow of liquid, and then intravenously administering the agent to the patient followed by reinserting the intravenous system into the patient; (2) the agent is added to the liquid in the container and then carried by the flow of the liquid to the patient; (3) agent is added to a liquid in a separate container called a partial fill that is connected to the primary intravenous line through which line the agent is carried by the flow of liquid to the patient; (4) an agent is contained in a piggyback vial into which is introduced an intravenous fluid, with the vital subsequently connected to the primary line through which the drug is administered to the patient; or (5) the agent is administered by a pump that exerts a force on a liquid containing agent for intravenously administering the liquid containing the agent. While these techniques are used, they have some disadvantages. For example, the administration of an agent through repeated insertion of a needle leads to unnecessary pain and trauma, they require separate connections for joining the primary intravenous line which further complicates intravenous administration, the use of pumps can produce pressures that can vary at the delivery site and the pressure can give rise to thrombosis, the rate of agent delivery to the patient often is unknown as it is not rate-controlled agent delivery but dependent on the flow of fluid, and they often require pre-formulation of the agent medication by the hospital pharmacist or nurse.

In the treatment of patients it is often desirable to provide a means whereby a drug or like agent can be introduced in a controlled manner over an extensive period of time. With the conventional intake of drugs and the like by means of periodic ingestion of a capsule or tablet, or periodic injection, there is usually an initial increase in the amount of the active agent in the body which is then reduced over a period of time, until the next tablet is taken or injection given.

In many cases it is preferable to provide an intake of drug in a controlled manner such that the amount of drug in the body remains substantially constant. This is particularly the case in the treatment of patients suffering constant pain as in the case of terminally ill patients who require a constant amount of an analgesic to be administered in order for them to be able to withstand the pain.

In the past several devices have been proposed for controlling the introduction of the drug into a body whereby it can be delivered in a controlled manner over a period of time. Such devices have comprised pumps driven by a power source, and which controls the delivery of drug to the body. Some of these pumps may be mounted externally to the body and are connected to a catheter introduced to the body of the patient. Other devices have comprised pump which is mounted subcutaneously to the body of the patient and which delivers a drug to the body at a desired location.

Other delivery devices have comprised a manually operated pump which may be mounted externally to the body or subcutaneously in the body of the patient whereby the pump can be activated by the patient for the delivery of the drug as need arises for that drug. Examples of such devices are disclosed in U.S. Pat. Nos. 4,588,394, 4,681,560 and, 5,085,644, and comprise devices whereby a pumping chamber is connected via catheter directly into the body and derives its source of drug from a holding reservoir.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

A. Effectors

SV28—Inositol hexaphosphate-cholesteroloxycarbonyl, hexa tributyl-ammonium salt.

SV184—Inositol hexaphosphate-cholesteroloxycarbonyl, undecanyl sod ium salt.

SV192—Inositol hexaphosphate-cholesteroloxycarbonyl, octa N,N-dimethyl-cyclohexylammonium salt.

KF16—Inositol hexaphosphate-cholesteroloxycarbonyl, hepta N,N-dimethyl-cyclohexylammonium salt.

B. Blood Preparations

Whole blood was collected from one subject. The blood was stored in a Vacutainer with Solution A (ACD) and stored at 4-8° C.

To isolate red blood cells, whole blood (3 mL) was placed on top of test tube containing 9 mL of Histopaque 1119 (Sigma Diagnostics Inc.) and 1 mL of Saline buffer. Following centrigugation the supernatant and buffy coat were removed and the pellet containing RBCs were washed three times in 10 mL HBS.

C. Buffers

HBS=HEPES Buffered Saline,

HBS was used as the standard buffer for experiments. HBS 7.42 (r.t.) was ideal to keep pH of experiments at 7.28-7.32 (37° C.).

20 mM HEPES

130 mM Sodium Chloride

HEPES, (N-[2-hydroxyethyl]piperazine-N′-[2-ethanesulfonic acid])

C₈H₁₈N₂O₄S

F.W.=238.3

pKₐ=7.5

pH: 6.8-8.2

CAS# 7365-45-9

HBS+HBS

20 μL Bovine Serum Albumin (BSA) per 5 mL HBS (TCS Medical Products Company)

15 μL Antifoaming per 5 mL HBS (TCS Medical Products Company)

pH: 7.2-7.4

Osmolarity: 290-320 mOsM

HBS·BSA 5 mL HBS Plus 20 μL BSA

saline, 0.9% Sodium Chlorida, Injection USP

Each 100 mL contains:

| | |
|---|---|
| 900 mg | NaCl |
| 154 mEq/L | Sodium |
| 154 mEq/L | Chloride |
| pH: | 5.0 |
| Osmolarity: | 308 mOsM |

BIS-TRIS buffered saline, (bis[2-Hydroxyethyl]minotris[hydroxymethyl]methane), (Sigma).

20 mM Bis-Tris

140 mM Sodium Chloride

pH: 7.45

Osmolarity: 294 mOsM

D. Procedures

Preparation of Effector Stock: Effector stock was prepared at 100-120 mM (Molal solution) using water or Bis-Tris Buffer. Effector characteristics prior to incubation were:

| | |
|---|---|
| Concentration: | 30 mM |
| Osmolarity: | 170-340 mOsM |
| pH: | 7.1-7.4 (at 37° C.) |

Incubation: Whole blood (75-300 μL) was incubated with 200 μL of effector at 37° C. for 5-10 min. (see Summary of Results below).

Washes: After incubation of whole blood with/without effector, blood cells were washed four times with Saline buffer or HBS (BSA) by centrifugal pelleting to remove exogenous effector and to evaluate hemolysis. After final centrifugation, pellet was not resuspended.

Blood Oxygen Dissociation Reading: Blood Oxygen Dissociation of samples were determined using a Hemox Analizer Model B (TCS Medical Products Company, New Hope, Pa.). The sample chamger contained:

Control:

2.5-3.0 mL of HBS+

25 μL Whole blood

Effector evaluation:

2.5-3.0 mL of HBS+

10-20 μL Pelleted Blood Cells incubated with Effector

All readings were made at 36.7-37.2° C. and at pH 7.28-7.32. The P₅₀s were calculated from the Dissociation Curves compared to same day control P₅₀.

E. Chemistry

IHP-Cholesterol Synthesis

Procedure—The IHP-ammonium salt was dissolved in anhydrous CH₂Cl₂/1,4-dioxane (5/2 mixture, final concentration=17 mM). Then, cholesterol chloroformate was added (1.1 eq.) and the reaction mixture was stirred 5 days under argon at room temperature. The solution was then concentrated in vacuo. The resulting white solid was triturated twice with hexane, the supernatants were separated to eliminate the residual chloroformate and traces of cholestrerol. No further purification was applied. The purity of the final compounds was assessed by HPLC.

HPLC conditions—Column: C18-reverse phase (Eclipse XDB-C18 4.6 mm ID/250 mm, 5 μm). Injection volume: 20 μl. Flow rate 1 ml.min⁻¹. Elution: CH₃CN/H₂O 4/1. Retention times: IHP (6.5 min.); IHP-Chol (9.5 min); IHP-(Chol)₂ (11.8 min).

IHP-Cholesterol prodrugs—From the optimized procedure, two different salts have been obtained: the first one with tributylammoniums countercations (compound SV28) the second one with N,N-dimethyl-cyclohexylammoniums (compounds SV192).

SV28 Inositol Hexaphosphate-cholesteroloxycarbonyl, Hexa Tributyl-ammonium Salt

White solid.

¹H-NMR (300 MHz, CDCl₃) δ 5.35 (d, J=5.0 Hz, 1H), 4.61 (m, 2H), 4.34 (m, 3H), 2.95 (bs, 36H), 2.1-1.0 (m, 131H), 0.64 (s, 3H).

³¹P-NMR (121 MHz, CDCl₃) δ non-acylatedphosphates 1.55, 1.13, 0.81, 0.66, 0.57, −0.38, −0.81, −0.96 (global integration: 5P), acylatedphosphates −6.28, −6.63, −7.35, −8.65, −10.98, −11.42, −11.45, −11.84, −14.89 (global integration: 1P).

Mass: (FAB−): M_{IHP}1=658.8 (40%), M_{IHP-chol}−1=1071.1 (60%), M_{IHP-(Chol)2}−1=1483.4 (10%).

HPLC (retention times, percentage integration) IHP (6.5 min, 1.8%); IHP-Chol (9.5 min, 86.5%); IHP-(Chol)₂ (11.8 min, 11.7%).

SV192 Inositol Hexaphosphate-cholesteroloxycarbonyl, Octa N,N-dimethylammonium Salt

White solid.

¹H-NMR (300 MHz, CDCl₃) δ 5.35 (d, J=5.0 Hz, 1H), 4.61 (m, 2H), 4.34 (m, 3H), 3.05 (m, 8H), 2.81 (s, 48H), 2.2-0.9 (m, 125H), 0.68 (s, 3H).

³¹P-NMR (121 MHz, CDCl₃) δ non-acylatedphosphates 1.55, 1.13, 0.81, 0.66, 0.57, −0.38, −0.81, −0.96 (global integration: 5P), acylatedphosphates −6.28, −6.63, −7.35, −8.65, −10.98, −11.42, −11.45, −11.84, −14.89 (global integration: IP).

F. Results

See Table 1.

### G. CONCLUSION

Prodrugs in which phytic acid has been covalently bound to a sterol moiety address the problem of transporting phytic acid (IHP) across the erythrocyte membrane. The covalent bond is a mixed anhydride susceptible to hydrolysis in vivo. The IHP-cholesterol derivative with N,N-dimethyl-cyclohexylammonium countercations displayed desirable biological properties. For example, the test performed on whole blood showed a shift of the O₂-dissociation curve without any detectable cell lysis.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are encompassed by the following claims.

We claim:

1. A composition, comprising an aliphatic ammonium cation, wherein the aliphatic ammonium cation is a lipophilic, water-soluble aliphatic ammonium cation; and a phosphorylated inositol or a phosphorylated glyceric acid covalently bound to a sterol moiety.

2. The composition of claim 1, wherein inositol hexaphosphate or 2,3-DPG is covalently bound to the sterol moiety.

3. The composition of claim 1, wherein inositol hexaphosphate is covalently bound to the sterol moiety.

4. The composition of claim 1, wherein the aliphatic ammonium cation is a monoalkyl, dialkyl, trialkyl or tetraalkyl ammonium moiety.

5. The composition of claim 1, wherein the aliphatic ammonium cation is an N,N-dimethyl-cyclohexylammonium cation.

6. The composition of claim 1, wherein the aliphatic ammonium cation is a monoalkyl ammonium cation.

7. The composition of claim 1, wherein the aliphatic ammonium cation is a primary ammonium cation.

8. The composition of claim 1, wherein the sterol moiety is a cholesterol moiety.

9. The composition of claim 1, wherein inositol hexaphosphate is covalently bound to the sterol moiety; and the sterol moiety is a cholesterol moiety.

10. A compound represented by structure 1: 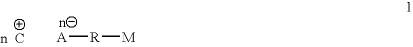
wherein
C⁺ represents independently for each occurrence an aliphatic ammonium cation, an alkali metal cation, or an alkaline earth cation; provided that at least one instance of C⁺ represents an aliphatic ammonium cation;
Aⁿ⁻ represents an anionic ligand for a mammalian cellular receptor;
R is an oxycarbonyl (—C(O)O—) linking group;
M is a lipophilic moiety; and
n is an integer in the range 1 to 11 inclusive.

11. The compound of claim 10, wherein an instance of C⁺ that represents an ammonium ion selected independently for each occurrence from the group consisting of C₁-C₆ alkyl ammonium ions and C₃-C₆ cycloalkyl ammonium ions.

12. The compound of claim 10, wherein an instance of C⁺ that represents an ammonium ion selected independently for each occurrence from the group consisting of C₃-C₆ cycloalkyl ammonium ions.

13. The compound of claim 10, wherein an instance of C⁺ that represents an ammonium ion selected independently for each occurrence from the group consisting of cyclohexyl ammonium ions.

14. The compound of claim 10, wherein an instance of C⁺ that represents an ammonium ion is N,N-dimethyl-cyclohexylammonium.

15. The compound of claim 10, wherein Aⁿ⁻ is a ligand for the allosteric site of hemoglobin.

16. The compound of claim 10, wherein Aⁿ⁻ is a phosphorylated inositol or a phosphorylated glyceric acid.

17. The compound of claim 10, wherein Aⁿ⁻ is a phosphorylated inositol or a phosphorylated glyceric acid, wherein said phosphorylated inositol or phosphorylated glyceric acid is a ligand for the allosteric site of hemoglobin.

18. The compound of claim 10, wherein Aⁿ⁻ is IHP or 2,3-DPG.

19. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a ligand for the allosteric site of hemoglobin.

20. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a ligand for the allosteric site of hemoglobin.

21. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid.

22. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a phosphorylated inositol or a phosphorylated glyceric acid.

23. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

24. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

25. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; and Aⁿ⁻ is IHP or 2,3-DPG.

26. The compound of claim 10, wherein an instance of C⁺ that represents an ammonium ion selected independently for each occurrence from the group consisting of C₁-C₆ alkyl ammonium ions and C₃-C₆ cycloalkyl ammonium ions; M is a cholesterol moiety; and Aⁿ⁻ is IHP or 2,3-DPG.

27. The compound of claim 10, wherein an instance of C⁺ that represents an ammonium ion selected independently for each occurrence from the group consisting of C₃-C₆ cycloalkyl ammonium ions; and Aⁿ⁻ is a ligand for the allosteric site of hemoglobin.

28. The compound of claim 10, wherein an instance of C⁺ that represents an ammonium ion selected independently for each occurrence from the group consisting of C₃-C₆ cycloalkyl ammonium ions; M is a cholesterol moiety; and Aⁿ⁻ is a ligand for the allosteric site of hemoglobin.

29. The compound of claim 10, wherein an instance of C⁺ that represents an ammonium ion selected independently for each occurrence from the group consisting of C₃-C₆ cycloalkyl ammonium ions; and Aⁿ⁻ is a phosphorylated inositol or a phosphorylated glyceric acid.

30. The compound of claim 10, wherein an instance of C⁺ that represents an ammonium ion selected independently for each occurrence from the group consisting of C₃-C₆ cycloalkyl ammonium ions; M is a cholesterol moiety; and Aⁿ⁻ is a phosphorylated inositol or a phosphorylated glyceric acid.

31. The compound of claim 10, wherein an instance of C⁺ that represents an ammonium ion selected independently for each occurrence from the group consisting of C₃-C₆ cycloalkyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

32. The compound of claim 10, wherein an instance of C⁺ that represents an ammonium ion selected independently for each occurrence from the group consisting of C₃-C₆ cycloalkyl ammonium ions; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

33. The compound of claim 10, wherein an instance of C⁺ that represents an ammonium ion selected independently for each occurrence from the group consisting of C₃-C₆ cycloalkyl ammonium ions; and Aⁿ⁻ is IHP or 2,3-DPG.

34. The compound of claim 10, wherein an instance of C⁺ that represents an ammonium ion selected independently for each occurrence from the group consisting of C₃-C₆ cycloalkyl ammonium ions; M is a cholesterol moiety; and Aⁿ⁻ is IHP or 2,3-DPG.

35. The compound of claim 10, wherein an instance of C⁺ that represents an ammonium ion selected independently for each occurrence from the group consisting of cyclohexyl ammonium ions; and Aⁿ⁻ is a ligand for the allosteric site of hemoglobin.

36. The compound of claim 10, wherein an instance of C⁺ that represents an ammonium ion selected independently for each occurrence from the group consisting of cyclohexyl ammonium ions; M is a cholesterol moiety; and Aⁿ⁻ is a ligand for the allosteric site of hemoglobin.

37. The compound of claim 10, wherein an instance of C⁺ that represents an ammonium ion selected independently for each occurrence from the group consisting of cyclohexyl ammonium ions; and Aⁿ⁻ is a phosphorylated inositol or a phosphorylated glyceric acid.

38. The compound of claim 10, wherein an instance of C⁺ that represents an ammonium ion selected independently for each occurrence from the group consisting of cyclohexyl ammonium ions; M is a cholesterol moiety; and Aⁿ⁻ is a phosphorylated inositol or a phosphorylated glyceric acid.

39. The compound of claim 10, wherein an instance of C⁺ that represents an ammonium ion selected independently for each occurrence from the group consisting of cyclohexyl ammonium ions; and Aⁿ⁻ is a phosphorylated inositol or a phosphorylated glyceric acid, wherein said phosphorylated inositol or phosphorylated glyceric acid is a ligand for the allosteric site of hemoglobin.

40. The compound of claim 10, wherein an instance of C⁺ that represents an ammonium ion selected independently for each occurrence from the group consisting of cyclohexyl ammonium ions; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

41. The compound of claim 10, wherein an instance of C⁺ that represents an ammonium ion selected independently for each occurrence from the group consisting of cyclohexyl ammonium ions; and Aⁿ⁻ is IHP or 2,3-DPG.

42. The compound of claim 10, wherein an instance of C⁺ that represents an ammonium ion selected independently for each occurrence from the group consisting of cyclohexyl ammonium ions; M is a cholesterol moiety; and Aⁿ⁻ is IHP or 2,3-DPG.

43. The compound of claim 10, wherein C⁺ is a sodium ion; M is a cholesterol moiety; and Aⁿ⁻ is a ligand for the allosteric site of hemoglobin.

44. The compound of claim 10, wherein C⁺ is a sodium ion; M is a cholesterol moiety; and Aⁿ⁻ is a phosphorylated inositol or a phosphorylated glyceric acid, wherein said phosphorylated inositol or phosphorylated glyceric acid is a ligand for the allosteric site of hemoglobin.

45. The compound of claim 10, wherein C⁺ is a sodium ion; M is a cholesterol moiety; and Aⁿ⁻ is IHP or 2,3-DPG.

46. A pharmaceutical formulation, comprising a composition of claim 1 or a compound of claim 12; and a pharmaceutically acceptable excipient.

47. A method of enhancing oxygen delivery to a tissue or organ of a mammal, comprising the step of administering to a mammal a therapeutically effective amount of a composition of claim 1 or a compound of claim 10.

48. A method of enhancing oxygen delivery to a tissue or organ of a mammal, comprising the step of administering to a mammal red blood cells previously treated with a composition of claim 1 or a compound of claim 10.

49. A method of treating a mammal afflicted with anemia, coronary infarction, pulmonary disease, congestive heart failure, diabetes, myocardial infarction, stroke, peripheral vascular disease, intermittent claudication, circulatory shock, hemorrhagic shock, chronic hypoxia, altitude sickness, arteriosclerosis, respiratory alkalemia, metabolic alkalosis, sickle cell anemia, reduced lung capacity, gangrene, anaerobic infections, carbon monoxide poisoning, nitric oxide poisoning, or cyanide poisoning, comprising the step of administering to said mammal a therapeutically effective amount of a composition of claim 1 or a compound of claim 10.

50. A method of treating a mammal afflicted with anemia, coronary infarction, pulmonary disease, congestive heart failure, diabetes, myocardial infarction, stroke, peripheral vascular disease, intermittent claudication, circulatory shock, hemorrhagic shock, chronic hypoxia, altitude sickness, arteriosclerosis, respiratory alkalemia, metabolic alkalosis, sickle cell anemia, reduced lung capacity, gangrene, anaerobic infections, carbon monoxide poisoning, nitric oxide poisoning, or cyanide poisoning, comprising the step of administering to said mammal red blood cells previously treated with a composition of claim 1 or a compound of claim 10.

51. A method of improving the oxygen delivering capability of mammalian blood, comprising the step of adding to a volume of mammalian blood a composition of claim 1 or a compound of claim 10.

52. A method of incorporating a therapeutically useful substance into mammalian red blood cells, comprising the step of treating a sample of mammalian red blood cells with a composition of claim 1 or a compound of claim 10, wherein said composition or compound comprises said therapeutically useful substance.
